(19) United States
(12) Patent Application Publication
(10) Pub. No.: US 2015/0072409 A1
(43) Pub. Date: Mar. 12, 2015

(54) CARTRIDGE FOR NUCLEIC ACID AMPLIFICATION REACTION

(71) Applicant: Seiko Epson Corporation, Tokyo (JP)

(72) Inventors: Yuji SAITO, Shiojiri (JP); Fumio TAKAGI, Chino (JP)

(21) Appl. No.: 14/478,328

(22) Filed: Sep. 5, 2014

(30) Foreign Application Priority Data
Sep. 6, 2013 (JP) 2013-185679

(51) Int. Cl. C12Q 1/68

(52) U.S. Cl. CPC C12Q 1/6806
USPC 435/293.1

(57) ABSTRACT

A cartridge for nucleic acid amplification reaction includes a tube including, on the inside, in order, a first plug formed by first oil, a second plug formed by a cleaning solution, which causes phase separation when mixed with oil, a third plug formed by second oil, a fourth plug formed by an eluate, which causes phase separation when mixed with oil, and a fifth plug formed by the third oil, a container for nucleic acid amplification reaction communicating with the fifth plug side of the tube and including fourth oil, and a plunger attached to an opening section on the first plug side of the tube and configured to push out liquid from the fifth plug side of the tube to the container for nucleic acid amplification reaction. All of the first to third oils have specific gravities different from the specific gravity of the fourth oil.

### BACKGROUND

1. Technical Field

The present invention relates to a cartridge for nucleic acid amplification reaction.

2. Related Art

Boom et al. reported a method of easily extracting nucleic acid from a biological material using a nucleic acid binding solid-phase carrier such as a silica particle and a chaotropic agent (see J. Clin. Microbiol., vol. 28 No. 3, p. 495 to 503 (1990)(Non Patent Literature 1)). A method of causing nucleic acid to adhere to a carrier and extracting the nucleic acid using the nucleic acid binding solid-phase carrier such as silica and the chaotropic agent including the method by Boom et al. mainly includes three steps: (1) a step of causing nucleic acid to adhere to the nucleic acid binding solid-phase carrier under the presence of the chaotropic agent (a adhering step), (2) a step of cleaning the carrier, to which the nucleic acid adheres, with a cleaning solution in order to remove nonspecifically-bound impurities and the chaotropic agent (a cleaning step), and (3) a step of eluting the nucleic acid from the carrier with water or low-salt concentration buffer solution (an eluting step).

Incidentally, in recent years, a thermal cycle device with easy control of a heating time has been developed other than a PCR device in the past (see Japanese Patent Application No. 2010-268090 (Patent Literature 1)). However, a cartridge and the like adaptable to these apparatuses have not been developed yet.

### SUMMARY

An advantage of some aspects of the invention is to provide a cartridge for nucleic acid amplification reaction that can easily cause nucleic acid amplification reaction.

An aspect of the invention is directed to a cartridge for nucleic acid amplification reaction including: a tube including, on the inside, in order, a first plug formed by first oil, a second plug formed by a first cleaning solution, which causes phase separation when mixed with oil and cleans a nucleic acid binding solid-phase carrier bound with nucleic acid, a third plug formed by second oil, a fourth plug formed by an eluate, which causes phase separation when mixed with oil and elutes the nucleic acid from the nucleic acid binding solid-phase carrier bound with the nucleic acid, and a fifth plug formed by third oil; and a container for nucleic acid amplification reaction communicating with the fifth plug side of the tube and including fourth oil on the inside. At least one of the first to third oils and the fourth oil have different specific gravities. The cartridge for nucleic acid amplification reaction may further include a plunger attached to an opening section on the first plug side of the tube and configured to push out liquid from the fifth plug side of the tube to the container for nucleic acid amplification reaction. On the inside of the plunger, fifth oil and a second cleaning solution, which causes phase separation when mixed with oil and cleans the nucleic acid binding solid-phase carrier bound with the nucleic acid may be stored. The eluate may contain a reagent that causes reverse transcription reaction. The eluate may contain a reagent that causes nucleic acid amplification reaction. The container for nucleic acid amplification reaction may include a seal forming section configured to fix the tube and a channel forming section in which droplets move. The seal forming section may include an oil receiving section configured to receive oil overflowing the channel forming section. The cartridge for nucleic acid amplification reaction may further include a tank communicating with the first plug side of the tube and configured to introduce the nucleic acid binding solid-phase carrier into the tube. The tank and the tube may be combined via the plunger.

In the cartridge for nucleic acid amplification reaction, it is preferable that the fourth oil has the specific gravity smaller than the specific gravities of all of the first to third oils.

In the cartridge for nucleic acid amplification reaction, it is preferable that, when the specific gravities of the first to third oils are respectively represented as A₁ to A₃ and the specific gravities of the first cleaning solution and the eluate are respectively represented as B₁ and B₂, concerning the plugs adjacent to each other and concerning at least one of A₁ to A₃ and at least one of B₁ and B₂, |Bm−An|≦0.12 (in the formula, n is an integer of 1 to 3 and m is 1 or 2) holds.

In the cartridge for nucleic acid amplification reaction, it is preferable that, when the specific gravity of the fourth oil is represented as A₄, |B₂−A₄≧0.06 holds.

In the cartridge for nucleic acid amplification reaction, it is preferable that the specific gravities of the first to third oils are equal to or larger than 0.88 and equal to or smaller than 1.10, the specific gravity of the fourth oil is equal to or larger than 0.80 and equal to or smaller than 0.95, and the specific gravities of the cleaning solution and the eluate are equal to or larger than 1.00 and equal to or smaller than 1.20.

Another aspect of the invention is directed to a cartridge kit for nucleic acid amplification reaction including: the cartridge for nucleic acid amplification reaction; and a tank configured to introduce the nucleic acid binding solid-phase carrier into the tube. The tank may include a solution for extracting nucleic acid and the nucleic acid binding solid-phase carrier. The tank may include an opening section. The opening section may include a removable lid. The opening section of the tank may be configured to be attachable to the opening section on the first plug side of the tube.

According to the aspects of the invention, it is possible to provide a cartridge for nucleic acid amplification reaction that can easily cause nucleic acid amplification reaction.

### BRIEF DESCRIPTION OF THE DRAWINGS

The invention will be described with reference to the accompanying drawings, wherein like numbers reference like elements.

FIGS. 1A and 1B are explanatory diagrams of a cartridge.

FIGS. 2A to 2C are operation explanatory diagrams of the cartridge.

FIGS. 3A to 3D are explanatory diagrams of a tank.

FIGS. 4A and 4B are explanatory diagrams of a fixing claw, a guide plate, and a mounting section.

FIGS. 5A and 5B are explanatory diagrams of the periphery of a PCR container.

FIG. 6A is a perspective view of the internal configuration of a PCR device.

FIG. 6B is a side view of the main configuration of the PCR device.

FIG. 7 is a block diagram of the PCR device.

FIG. 8A is an explanatory diagram of a rotating body.

FIG. 8B is an explanatory diagram of a state in which the cartridge is mounted on the mounting section of the rotating body.

FIGS. 9A to 9D are explanatory diagrams of states of the PCR device during the mounting of the cartridge.

FIG. 10 is a conceptual diagram of the behavior of magnetic beads at the time when a magnet is moved in a downward direction.

FIGS. 11A to 11C are explanatory diagrams of nucleic acid elution treatment.

FIG. 12 is a conceptual diagram of the behavior of the magnetic beads at the time when the magnet is swung.

FIG. 13 is a table showing presence or absence of the swing of the magnet.

FIGS. 14A to 14C are explanatory diagrams of droplet forming treatment.

FIGS. 15A to 15D are explanatory diagrams of thermal cycle treatment.

FIG. 16 is a diagram showing a kit for nucleic acid extraction and a device after assembly of the kit for nucleic acid extraction used in an embodiment of the invention.

FIG. 17 is a graph showing a result of real-time PCR in the embodiment of the invention.

FIG. 18 is a graph representing a result obtained by examining a relation with centrifugal force resistance concerning a specific gravity difference between a water solution plug and an oil plug in a capillary.

FIG. 19 is a graph representing a result obtained by examining a relation with a moving time of a droplet concerning a specific gravity difference between a water solution plug and an oil plug in the PCR container.

### DESCRIPTION OF EXEMPLARY EMBODIMENTS

An embodiment of the invention is explained in detail below. Note that an object, characteristics, advantages, and ideas of the invention are obvious for those skilled in the art from the description of this specification. Those skilled in the art can easily reproduce the invention from the description of this specification. The embodiment of the invention described below indicates a preferred implementation mode of the invention. The embodiment is described for illustration and explanation and does not limit the invention to the embodiment. It is obvious for those skilled in the art that various alternations and modifications can be made on the basis of the description of this specification within the intention and the scope of the invention disclosed in this specification.

First, a cartridge mounted on a PCR device 100 is explained. Then, the configuration and the operation of the PCR device 100 in this embodiment are explained.

### Cartridge

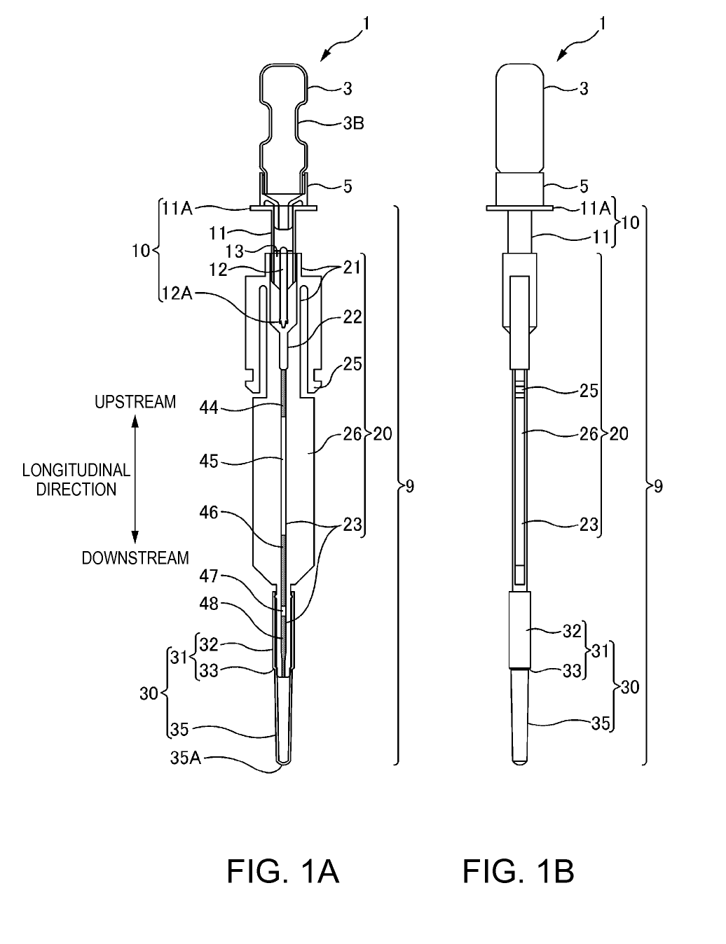

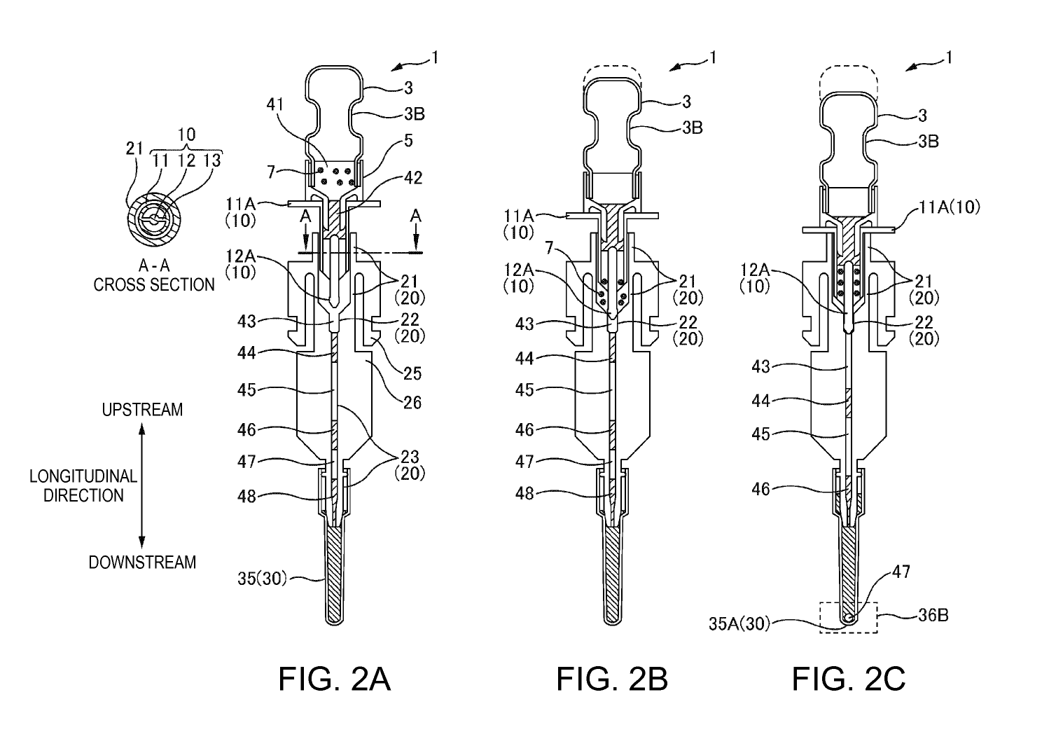

FIGS. 1A and 1B are explanatory diagrams of a cartridge 1. FIGS. 2A to 2C are operation explanatory diagrams of the cartridge 1. FIG. 2A is an explanatory diagram of an initial state of the cartridge 1. FIG. 2B is a side view of the cartridge 1 at the time when a plunger 10 is pushed in the state shown in FIG. 2A and a seal 12A comes into contact with a lower syringe 22. FIG. 2C is an explanatory diagram of the cartridge 1 after the plunger 10 is pushed.

The cartridge 1 is configured by a tank 3 and a cartridge main body 9. The cartridge 1 is a container for performing nucleic acid elution treatment for eluting nucleic acid from a nucleic acid binding solid-phase carrier 7 bound with the nucleic acid into a plug 47 used for polymerase reaction and is a container for applying thermal cycle treatment of the polymerase reaction to a reaction solution droplet 47.

Nucleic acid extraction treatment is performed in the tank 3. The nucleic acid is refined while the nucleic acid passes through a tube 20. The material of the tube 20 is not particularly limited but can be, for example, glass, resin such as plastics, or metal. In particular, it is preferable to select transparent glass or resin as the material of the tube 20 because the inside of a hollow can be observed from the outside of the tube 20. It is preferable to select a substance or a nonmagnetic body, which transmits a magnetic force, because, for example, when magnetic particles are caused to pass through the tube 20, this is facilitated by applying a magnetic force to the magnetic particles from the outside of the tube 20. Note that the material of the tube 20 may be the same as the material of the tank 3.

The tube 20 includes a first plug (a first oil plug) 44 formed by first oil, a second plug (a cleaning solution plug) 45 formed by a first cleaning solution, a third plug (a second oil plug) 46 formed by second oil, a third plug (a reaction solution plug) 47 formed by a reaction solution, and a fifth plug (a third oil plug) 48 formed by third oil. Magnetic beads 7 bound with nucleic acid are attracted to a magnet on the outside. Therefore, due to moving the magnet outside along the tube 20, the magnetic beads 7 move in the tube 20 and reach the reaction solution plug 47 through the cleaning solution plug 45. The nucleic acid bound to the magnetic beads 7 is cleaned by the cleaning solution in the cleaning solution plug 45 and eluted in the reaction solution plug 47. The “plug” means specific liquid when the specific liquid occupies a section in the tube 20. For example, liquid retained in a columnar shape in a capillary 23 in FIGS. 2A to 2C is referred to as “plug”. The “oil” means liquid not mixed with water and includes the first oil, the second oil, and the third oil explained above and fourth oil and fifth oil explained below. Therefore, the third plug formed by the second oil has a function of preventing water-soluble plugs on both sides of the third plug from being mixed with each other. It is preferable that air bubbles and other liquid are absent in the plugs or between the plugs. However, air bubbles and other liquid may be present as long as the magnetic beads 7 can pass through the plugs.

A type of the oil is not particularly limited. Mineral oil, silicone oil (2CS silicone oil, etc.), vegetable oil, and the like can be used. However, by using oil having higher viscosity, it is possible to improve a “wiping effect” by the oil when the nucleic acid binding solid-phase carrier is moved on the interface between the oil and the plug on the upper side. Consequently, when the nucleic acid binding solid-phase carrier is moved from the plug on the upper side to the plug formed by the oil, it is possible to make it difficult to bring a water-soluble component adhering to the nucleic acid binding solid-phase carrier into the oil.

The thermal cycle treatment is performed in the PCR container 30 of the cartridge 1. The PCR container 30 is filled with the fourth oil. The reaction solution causes phase separation when mixed with the fourth oil. Therefore, when the reaction solution plug 47 is pushed out into the PCR container 30 from the tube 20, the reaction solution plug 47 changes to a droplet form. Since the reaction solution plug 47 has specific gravity larger than the specific gravity of the fourth oil, the reaction solution droplet 47 precipitates. A high-temperature region 36A and a low-temperature region 36B are formed in the PCR container 30 by a heater on the outside. The entire cartridge 1 is vertically reversed together with the heater. When this is repeated, the reaction solution droplet 47 alternately moves between the high-temperature region 36A and the low-temperature region 36B. Temperature treatment in two stages is applied to the reaction solution droplet 47.

The material of the PCR container 30 is not particularly limited. However, the material can be, for example, glass, resin such as plastics, or metal. Since a high-temperature side heater 65B is present near the PCR container 30, it is preferable that the material of the PCR container 30 has at least heat resistance to temperature equal to or higher than 100° C. It is preferable to select a transparent or semitransparent material as the material of the PCR container 30 because it is easy to perform fluorescence measurement (luminance measurement). However, the entire region of the PCR container 30 does not need to be transparent or semitransparent. At least a region (e.g., a bottom 35A of the PCR container 30) opposed to a fluorescence measuring instrument 55 only has to be transparent or semitransparent. Note that the material of the tube 20 may be the same as the material of the tank 3 and the plunger 10.

The cartridge 1 is configured by the tank 3 and the cartridge main body 9. In a kit forming the cartridge 1, an adapter 5 is prepared in advance together with the tank 3 and the cartridge main body 9. The tank 3 and the cartridge main body 9 are connected via the adapter 5, whereby the cartridge 1 is assembled. However, the tank 3 can also be configured to be directly attached to the cartridge main body 9.

In the following explanation of the components of the cartridge 1, as shown in FIG. 2A, a direction along the long cartridge 1 is represented as “longitudinal direction), the tank 3 side is represented as “upstream side”, and the PCR container 3 side is represented as “downstream side”. Note that the upstream side is sometimes represented simply as “up” and the downstream side is sometimes represented simply as “down”.

### (1) Tank

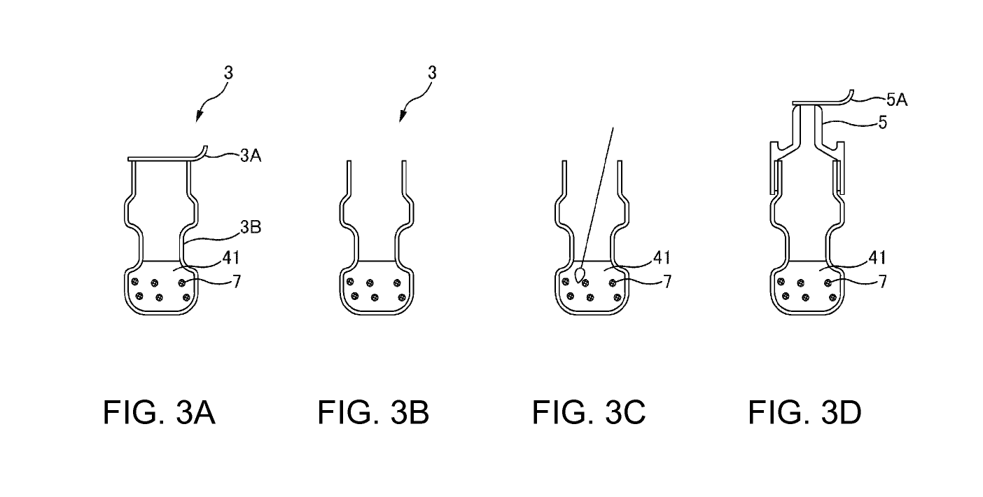

FIGS. 3A to 3D are explanatory diagrams of the tank 3.

In the tank 3 prepared in advance in the kit, a solution 41 and the nucleic acid binding solid-phase carrier, for example, the magnetic beads 7 are stored. A removable lid 3A is attached to an opening of the tank 3 (see FIG. 3A). As the solution 41, 5M of guanidine thiocyanate, 2% of Triton X-100, and 50 mM of Tris-HCl (pH 7.2) are used. An operator removes the lid 3A to open the opening of the tank 3 (see FIG. 3B), immerses a cotton swab with virus adhering thereto in the solution 41 in the tank 3, and collects the virus in the solution 41 (see FIG. 3C). When liquid in the tank 3 is agitated, the tank 3 may be shaken in a state shown in FIG. 3C. However, since the solution 41 tends to overflow, as shown in FIG. 3D, it is preferable to attach the adapter 5 having a lid 5A to the opening of the tank 3 and then shake the tank 3. Consequently, a substance in the tank 3 is agitated, virus particles are dissolved by the solution 41, nucleic acid is separated, and silica covering the magnetic beads 7 absorbs the nucleic acid. Thereafter, the operator removes the lid 5A of the adapter 5 attached to the opening of the tank 3 and attaches the tank 3 to the cartridge main body 9 via the adapter 5 (see FIG. 2A).

The tank 3 is formed of flexible resin. The tank 3 is expandable. When the plunger 10 slides and a state shown in FIG. 2A changes to a state shown in FIG. 2B, the tank 3 expands, whereby the pressure of the liquid in the tube 20 is suppressed from excessively increasing and the liquid in the tube 20 is suppressed from being pushed out to the downstream side. It is desirable to form a deformed section 3B in the tank 3 to allow the tank 3 to easily expand.

Note that a sample from which nucleic acid is extracted and amplified is not limited to the virus and may be a cell. Derivation of the cell is not particularly limited and may be a microorganism or may be a tissue piece, blood, or the like of a higher organism.

The solution is not particularly limited as long as the solution contains a chaotropic substance. However, a surface active agent may be contained for the purpose of destroying a cell membrane or degenerating protein contained in a cell. The surface active agent is not particularly limited as long as the surface active agent is generally used for extracting nucleic acid from a cell or the like. Specifically, examples of the surface active agent include nonionic surface active agents like a triton surface active agent such as Triton-X and a Tween surface active agent such as Tween20 and an anionic surface active agent such as N-sodium lauroylsarcosine (SDS). In particular, it is preferable to use the nonionic surface active agent in a range of 0.1 to 2%. Further, it is preferable that a reducing agent such as 2-mercaptoethanol or dithiothreitol is contained in the solution. The solution may be a buffer solution. It is preferable that the solution is neutral with pH 6 to 8. Taking these into account, specifically, it is preferable that 3 to 7 M of guanidine salt, 0 to 5% of a nonionic surface active agent, 0 to 0.2 mM of EDTA, 0 to 0.2 M of a reducing agent, or the like may be contained in the solution.

The chaotropic substance is not particularly limited as long as the chaotropic substance generates chaotropic ions (univalent anions having a large radius) in a water solution, has action of increasing water solubility of hydrophobic molecules, and contributes to adhesion of nucleic acid to a solid-phase carrier. Specifically, examples of the chaotropic substance include guanidine thiocyanate, guanidine hydrochloride, sodium iodide, potassium iodide, and sodium perchlorate. Among these substances, the guanidine thiocyanate or the guanidine hydrochloride having strong protein degeneration action is preferable. Concentration of use of the chaotropic substance is different depending on the substances. For example, when the guanidine thiocyanate is used, it is preferable to use the guanidine thiocyanate in a range of 3 to 5.5 M. When the guanidine hydrochloride is used, it is preferable to use the guanidine hydrochloride in a range equal to or higher than 5 M.

An instrument for collecting a sample is not particularly limited. Instead of the cotton swab, a spatula, a bar, a scraper, or the like only has to be selected according to a use.

An internal volume of the tank 3 is not particularly limited. However, the internal volume can be set to be equal to or larger than 0.1 mL and equal to or smaller than 100 mL. The material of the tank 3 is not particularly limited. However, the material of the tank 3 can be, for example, glass, resin such as plastics, or metal. In particular, it is preferable to select transparent glass or resin as the material of the tank 3 because the inside of the tank 3 can be observed from the outside. The tank 3 and the tubes 20 may be integrally molded or may be detachable or attachable. When a material having flexibility such as rubber, elastomer, or polymer is used as the material of the tank 3, in a state in which a lid is attached to the tank 3, the inside of the tank 3 can be pressurized by deforming the tank 3. Consequently, content of the tube 20 can be pushed out from the distal end side of the tube 20 to be pushed out from the inside to the outside of the tube 20.

### (2) Cartridge Main Body

The cartridge main body 9 includes the plunger 10, the tube 20, and the PCR container 30.

### (2-1) Plunger

The plunger 10 is explained below with reference to FIGS. 2A to 2C.

The plunger 10 is a movable plunger that pushes out liquid from the downstream side of the tube 20 functioning as a syringe. The plunger 10 has a function of pushing out a predetermined amount of liquid in the tube 20 from the terminal end of the tube 20 to the PCR container 30. The plunger 10 also has a function of attaching the tank 3 via the adapter 5.

The plunger 10 includes a cylindrical section 11 and a bar-like section 12. The cylindrical section 11 is provided on the tank 3 side (the upstream side). The bar-like section 12 is provided on the tube 20 side (the downstream side). The bar-like section 12 is supported by tabular two ribs 13 from the inner wall on the downstream side of the cylindrical section 11. The downstream side of the bar-like section 12 projects to the downstream side from the cylindrical section 11.

The cylindrical section 11 is opened to the upstream side and the downstream side. The inner wall of the cylindrical section 11 functions as a passage for liquid. The adapter 5 fits in the opening on the upstream side (the tank 3 side) of the cylindrical section 11. In the plunger 10 of the cartridge main body 9 prepared in advance in the kit, a detachable lid may be attached to the opening on the upstream side of the cylindrical section 11. The opening on the downstream side of the cylindrical section 11 is located on the inside of an upper syringe 21 of the tube 20. The magnetic beads 7 introduced from the opening on the upstream side of the cylindrical section 11 pass the inside of the cylindrical section 11, pass through the front and the back of the ribs 13, and exit from the opening on the downstream side of the cylindrical section 11 to be introduced into the upper syringe 21 of the tube 20.

The downstream side of the cylindrical section 11 fits with the inner wall of the upper syringe 21 of the tube 20. The cylindrical section 11 can slide in the longitudinal direction with respect to the upper syringe 21 of the tube 20 while being inscribed in the upper syringe 21.

An attachment stand 11A, to which the adapter 5 is attached, is formed around the opening on the upstream side of the cylindrical section 11. The attachment stand 11A is also apart that is pushed when the plunger 10 is pushed. When the attachment stand 11A is pushed, the plunger 10 slides with respect to the tube 20 and the state shown in FIG. 2A changes to the state shown in FIG. 2C. When the plunger 10 moves to the downstream side, the attachment stand 11A comes into contact with the upper edge of the tube 20 (see FIG. 2C). That is, an interval between the attachment stand 11A of the plunger 10 and the upper edge of the tube 20 is a slide length of the plunger 10.

In an initial state, the bar-like section 12 is located on the inside of the upper syringe 21 of the tube 20 and separated from the lower syringe 22 (see FIG. 2A). When the plunger 10 slides with respect to the tube 20, the bar-like section 12 is inserted into the lower syringe 22 of the tube 20. The bar-like section 12 slides in the downstream direction with respect to the lower syringe 22 while being inscribed in the lower syringe 22 (see FIGS. 2B and 2C).

The shape of a cross section orthogonal to the longitudinal direction of the bar-like section 12 is a circular shape. However, a cross sectional shape of the bar-like section 12 can be a circle, an ellipse, or a polygon and is not particularly limited as long as the bar-like section 12 can fit with the inner wall of the lower syringe 22 of the tube 20.

The seal 12A is formed at an end on the downstream side of the bar-like section 12. When the seal 12A fits in the lower syringe 22, the liquid in the tube 20 on the downstream side is prevented from flowing back to the upper syringe 21. When the plunger 10 is pushed from the state shown in FIG. 2B to the state shown in FIG. 2C, the liquid in the tube 20 is pushed out from the downstream side by an amount equivalent to a volume of the slide of the seal 12A in the lower syringe 22 during the pushing of the plunger 10.

Note that the volume of the slide of the seal 12A in the lower syringe 22 (the amount of the liquid in the tube 20 pushed out from the downstream side) is larger than a total of the reaction solution plug 47 and the third oil plug 48 in the tube 20. Consequently, it is possible to push out the liquid in the tube 20 without leaving the reaction solution in the tube 20.

The material of the plunger 10 is not particularly limited. However, the material can be, for example, glass, resin such as plastics, or metal. The cylindrical section 11 and the bar-like section 12 of the plunger 10 may be integrally formed of the same material or may be formed of different materials. In this embodiment, the cylindrical section 11 and the bar-like section 12 are separately molded with resin. The cylindrical section 11 and the bar-like section 12 are joined via the ribs 13, whereby the plunger 10 is formed.

On the inside of the plunger 10, oil 42 and a second cleaning solution 43 are stored in advance. The oil 42 in the plunger 10 has specific gravity smaller than the specific gravity of the second cleaning solution 43. Therefore, when the cartridge main body 9 is erected with the attachment stand 11A of the plunger 10 facing up when the tank 3 is attached to the cartridge main body 9, as shown in FIG. 2A, the oil 42 is arranged between the liquid in the tank 3 and the second cleaning solution 43 of the cartridge main body 9. As the oil 42, 2CS silicon oil is used. As the second cleaning solution 43, 8M of guanidine hydrochloride or 0.7% of Triton X-100 is used.

Note that the second cleaning solution 43 only has to be liquid that causes phase separation when mixed with both of the oil 42 and the oil 44 that forms the first oil plug. The second cleaning solution 43 is preferably water or a low-salt concentration water solution. In the case of the low-salt concentration water solution, the low-salt concentration water solution is preferably a buffer solution. The salt concentration of the low-salt concentration water solution is preferably equal to or lower than 100 mM, more preferably equal to or lower than 50 mM, and most preferably equal to or lower than 10 mM. There is no particular limit of the low-salt concentration water solution. However, the lower limit is preferably equal to or higher than 0.1 mM, more preferably equal to or higher than 0.5 mM, and most preferably equal to or higher than 1 mM. The solution may contain a surface active agent such as Triton, Tween, or SDS and pH of the solution is not particularly limited. Salt for changing the solution to a buffer solution is not particularly limited. However, salt such as Tris, Hepes, Pipes, or phosphoric acid is preferably used. Further, the cleaning solution preferably contains alcohol by an amount that does not inhibit adhesion of nucleic acid to a carrier, reverse transcription reaction, PCR reaction, and the like. In this case, alcohol concentration is not particularly limited. However, the alcohol concentration may be equal to or lower than 70%, may be equal to or lower than 60%, may be equal to or lower than 50%, may be equal to or lower than 40%, may be equal to or lower than 30%, may be equal to or lower than 20%, or may be equal to or lower than 10%. However, the alcohol concentration is preferably equal to or lower than 5% or equal to or lower than 2%, more preferably equal to or lower than 1% or equal to or lower than 0.5%, and most preferably equal to or lower than 0.2% or equal to or lower than 0.1%.

Note that a chaotropic agent may be contained in the second cleaning solution 43. For example, when guanidine hydrochloride is contained in the second cleaning solution 43, it is possible to clean particles and the like while maintaining or intensifying adhesion of nucleic acid adhering to the particles and the like. When the guanidine hydrochloride is contained, the concentration of the guanidine hydrochloride can be set to be, for example, equal to or higher than 3 mol/L and equal to or lower than 10 mol/L and, preferably, equal to or higher than 5 mol/L and equal to or lower than 8 mol/L. If the concentration of the guanidine hydrochloride is in this range, it is possible to clean other impurities and the like while causing the nucleic acid adhering to the particles and the like to more stably adhere to the particles and the like.

### (2-2) Tube

The tube 20 is explained below with reference to FIGS. 2A to 2C.

The tube 20 has a cylindrical shape that can circulate liquid in the longitudinal direction. The tube 20 includes the upper syringe 21, the lower syringe 22, and the capillary 23. The inner diameters of the sections are varied stepwise.

The upper syringe 21 has a cylindrical shape that can circulate liquid in the longitudinal direction. The cylindrical section 11 of the plunger 10 is slidably inscribed in the inner wall of the upper syringe 21. The upper syringe 21 functions as a syringe for the cylindrical section 11 of the plunger 10.

The lower syringe 22 has a cylindrical shape that can circulate liquid in the longitudinal direction. The seal 12A of the bar-like section 12 of the plunger 10 can slidably fit with the inner wall of the lower syringe 22. The lower syringe 22 functions as a syringe for the bar-like section 12 of the plunger 10.

The capillary 23 has a thin tube-like shape that can circulate liquid in the longitudinal direction. The inner diameter of the capillary 23 has size that allows the liquid to maintain the shape of a plug. The inner diameter is 1.0 mm. At the terminal end of the capillary 23 (the end on the downstream side of the tube 20), the inner diameter is smaller than 1.0 mm. The inner diameter at the terminal end of the capillary 23 is 0.5 mm. The inner diameter at the terminal end of the capillary 23 is set smaller than the diameter (1.5 to 2.0 mm) of a droplet-like reaction solution explained below. Consequently, when the reaction solution plug 47 is pushed out from the terminal end of the capillary 23, it is possible to prevent the droplet-like reaction solution from adhering to the terminal end of the capillary 23 or reversely flowing into the capillary 23.

Note that the capillary 23 only has to be a capillary including a hollow on the inside and having a cylindrical shape that can circulate liquid in the longitudinal direction. The capillary 23 may be bent in the longitudinal direction. However, the capillary 23 is preferably linear. Both of the size and the shape of the hollow on the inside of the tube 20 are not particularly limited as long as the liquid can maintain the shape of the plug in the tube 20. The size of the hollow in the tube 20 and the shape of the cross section perpendicular to the longitudinal direction may change according to the longitudinal direction of the tube 20.

The shape of the cross section perpendicular to the longitudinal direction of the external shape of the tube 20 is not limited either. The thickness of the tube 20 (the length from the side surface of the hollow on the inside to the surface on the outside) is not particularly limited either. When the tube has a cylindrical shape, the inner diameter of the tube 20 (the diameter of a circle in the cross section perpendicular to the longitudinal direction of the hollow on the inside) can be set to be, for example, equal to or larger than 0.5 mm and equal to or smaller than 2 mm. When the inner diameter of the tube 20 is in this range, a plug of liquid is easily formed in a wide range in terms of the material of the tube 20 and a type of the liquid. The distal end is preferably reduced in a taper shape and can be set to be equal to or larger than 0.2 mm and equal to or smaller than 1 mm. By setting the inner diameter of the terminal end of the capillary 23 (an opening diameter of the capillary 23) small, it is possible to suppress the reaction solution droplet 47 from adhering to and not separating from the opening of the capillary 23. However, if the inner diameter of the terminal end of the capillary 23 is set excessively small, a large number of small reaction solution droplets 47 are formed. Note that it is undesirable to reduce the diameter of parts other than the terminal end of the capillary 23 in the same manner as the terminal end because the cartridge 1 is formed long from the necessity of securing the volumes of the plugs.

The capillary 23 includes, in order from the upstream side, the first oil plug 44, the cleaning solution plug 45, the second oil plug 46, the reaction solution plug 47, and the third oil plug 48 on the inside. That is, the oil plugs are arranged on both sides of the water-soluble plug (the cleaning solution plug 45 or the reaction solution plug 47).

Note that the oil 42 and the cleaning solution 43 are stored in advance in the upper syringe 21 and the lower syringe 22 further on the upstream side than the first oil plug 44 (see FIG. 2A). The inner diameter of the upper syringe 21 and the lower syringe 22 is larger than the inner diameter of the capillary 23. The liquid (the oil 42 and the cleaning solution 43) cannot be maintained in a columnar shape like a plug in the upper syringe 21 and the lower syringe 22. However, since the first oil plug 44 is retained in the shape of the plug by the capillary 23, the oil forming the first oil plug 44 is suppressed from moving to the upstream side.

The cleaning solution plug 45 may be formed of 5 mM of a tris-hydrochloric acid buffer solution. However, the cleaning solution may basically have composition same as the composition explained about the second cleaning solution. The cleaning solution may be the same as or different from the second cleaning solution. However, the cleaning solution is preferably a solution not practically containing a chaotropic substance. This is for the purpose of preventing the chaotropic substance from being carried into a later solution. As explained above, the cleaning solution preferably contains alcohol by an amount not inhibiting adhesion of nucleic acid to a carrier, reverse transcription reaction, PCR reaction, and the like. In this case, alcohol concentration is not particularly limited. However, the alcohol concentration may be equal to or lower than 70%, may be equal to or lower than 60%, may be equal to or lower than 50%, may be equal to or lower than 40%, may be equal to or lower than 30%, may be equal to or lower than 20%, or may be equal to or lower than 10%. However, the alcohol concentration is preferably equal to or lower than 5% or equal to or lower than 2%, more preferably equal to or lower than 1% or equal to or lower than 0.5%, and most preferably equal to or lower than 0.2% or equal to or lower than 0.1%.

The cleaning solution plug 45 may be formed from a plurality of plugs divided by plugs of oil. When the cleaning solution plug 45 is formed from a plurality of plugs, liquids of the plugs may be the same or may be different. If at least one plug of a cleaning solution is present among the plugs, the liquids of the other plugs are not particularly limited. However, it is preferable that all the plugs are cleaning solution. The number of division of the cleaning solution plug 45 can be set as appropriate taking into account, for example, the length of the tube 20, a target of cleaning, and the like.

The reaction solution plug 47 is formed by a reaction solution. The reaction solution means liquid that elutes nucleic acid adhering to a nucleic acid binding solid-phase carrier from the carrier into the liquid and causes reverse transcription reaction and polymerase reaction. Therefore, the reaction solution after the elution of the nucleic acid is prepared in advance to be a buffer solution directly used in the reverse transcription reaction and the polymerase reaction.

The reaction solution may contain, for the reverse transcription reaction, reverse transcriptase, dNTP, and a primer (oligonucleotide) for the reverse transcriptase. The reaction solution may further contain, for the polymerase reaction, DNA polymerase and a primer (oligonucleotide) for DNA polymerase and include a probe for real-time PCR such as a TaqMan probe, a Molecular Beacon, or a cycling probe and a fluorescent dye for intercalator such as SYBR green. Further, the reaction solution preferably further contains, as a reaction inhibition preventing agent, BSA (bovine serum albumin) or gelatin. A solvent is preferably water and more preferably a solvent not practically containing organic solvents such as ethanol and isopropyl alcohol and a chaotropic substance. The reaction solution preferably contains salt to be a buffer solution for reverse transcriptase and/or a buffer solution for DNA polymerase. The salt for changing the reaction solution to the buffer solution is not particularly limited as long as the salt does not inhibit enzyme reaction. However, salt such as Tris, Pepes, Pipes, or phosphoric acid is preferably used. The reverse transcriptase is not particularly limited. For example, reverse transcriptase deriving from Avian Myeloblast Virus, Ras Associated Virus Type 2, Mouse Molony Murine Leukemia Virus, or Human Immunodeficiency Virus Type 1 can be used. However, a heat resistant enzyme is preferable. The DNA polymerase is not particularly limited either. However, a heat resistant enzyme or an enzyme for PCR is preferable. For example, there are an extremely large number of commercially available products such as Taq polymerase, Tfi polymerase, Tth polymerase, and improvements of these polymerases. However, the DNA polymerase that can perform a hot start is preferable.

The concentration of the dNTP and the salt contained in the reaction solution only has to be set to concentration suitable for an enzyme in use. Usually, the concentration of the dNTP only has to be set to 10 to 1000 μM and preferably 100 to 500 μM, the concentration of Mg²⁺ only has to be set to 1 to 100 mM and preferably 5 to 10 mM, and the concentration of Cl⁻ only has to be set to 1 to 2000 mM and preferably 200 to 700 mM. Total ion concentration is not particularly limited. However, the total ion concentration may be concentration higher than 50 mM, preferably higher than 100 mM, more preferably higher than 120 mM, and sill more preferably higher than 150 mM, and still more preferably higher than 200 mM. An upper limit of the total ion concentration is preferably equal to or lower than 500 mM, more preferably equal to or lower than 300 mM, and still more preferably equal to or lower than 200 mM. The oligonucleotide for a primary is used by 0.1 to 10 μM and preferably 0.1 to 1 μM. When the concentration of the BSA or the gelatin is equal to or lower than 1 mg/mL, a reaction inhibition preventing effect is small. When the concentration of the BSA or the gelatin is equal to or higher than 10 mg/mL, it is likely that the BSA or the gelatin inhibits the reverse transcription and enzyme reaction after the reverse transcription. Therefore, 1 to 10 mg/mL is preferable. When the gelatin is used, as derivation of the gelatin, cowhide, pig hide, and cow bone can be illustrated but is not particularly limited. When the gelatin is not easily dissolved, the gelatin may be heated and dissolved.

For example, as the reaction solution, solutions described below can be used.

| | | |
|---|---|---|
| 0.2 | u/μL | AMV reverse transcriptase (Nippon Gene Co., Ltd.) |
| 0.125 | u/μL | Gene Taq NT PCR enzyme (Nippon Gene Co., Ltd.) |
| 0.5 | mM | dNTP |
| 1.0 | μM | Primer (forward) |
| 1.0 | μM | Primer (reverse) |
| 0.5 | μM | Probe (Taq man) |
| 4.0 | mg/mL | BSA |

| | |
|---|---|
| x1 | Buffer (MgCl₂ 7 mM; Tris pH 9.0 25 mM; KCl 50 mM) |

The volume of the reaction solution plug 47 is not particularly limited and can be set as appropriate using, for example, an amount of particles or the like, to which nucleic acid is caused to adhere, as an index. For example, when the volume of the particles or the like is 0.5 μL, it is sufficient if the volume of the reaction solution plug 47 is equal to or larger than 0.5 μl. The volume is preferably set to be equal to or larger than 0.8 μL and equal to or smaller than 5 μL, more preferably equal to or larger than 1 μL and equal to or smaller than 3 μL. If the volume of the reaction solution plug 47 is in these ranges, for example, even if the volume of the nucleic acid binding solid-phase carrier is set to 0.5 μL, it is possible to sufficiently elute nucleic acid from the carrier.

The downstream section of the capillary 23 is inserted into the PCR container 30. Consequently, it is possible to push out the reaction solution to the PCR container 30 by pushing out the reaction solution plug 47 in the tube 20 from the tube 20.

An annular convex section of the outer wall of the capillary 23 comes into contact with the inner wall of the PCR container 30, whereby an upper seal section is formed. The outer wall of the capillary 23 further on the downstream side than the upper seal section comes into contact with the inner wall of the PCR container 30, whereby a lower seal section is formed. The upper seal section and the lower seal section are explained below.

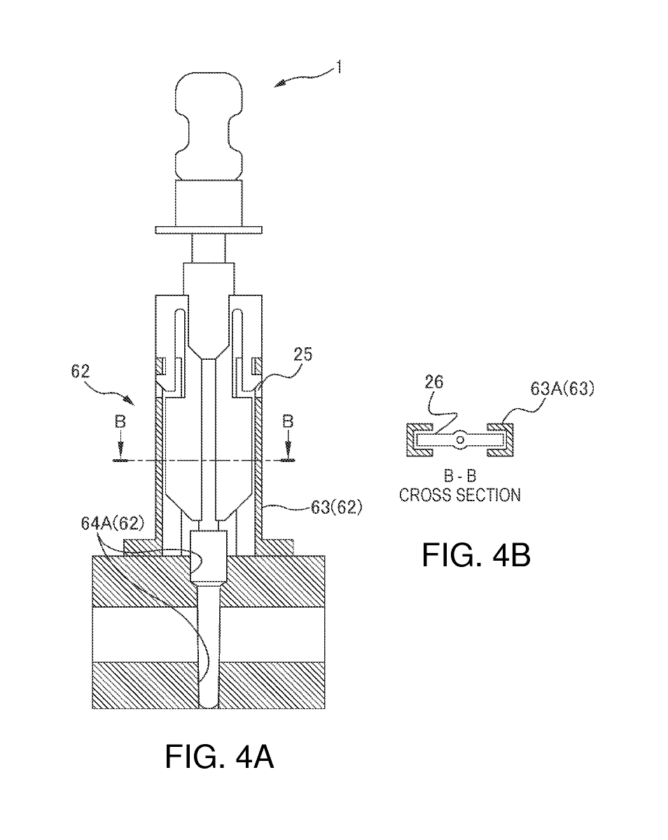

The tube 20 further includes a fixing claw 25 and a guide plate 26. FIGS. 4A and 4B are explanatory diagrams of the fixing claw 25 and the guide plate 26 and a mounting section 62.

The fixing claw 25 is a member that fixes the cartridge 1 to the mounting section 62. When the cartridge 1 is inserted into the mounting section 62 until the cartridge 1 is caught by the fixing claw 25, the cartridge 1 is fixed in a normal position with respect to the mounting section 62. In other words, when the cartridge 1 is present in an abnormal position with respect to the mounting section 62, the fixing claw 25 is not caught by the mounting section 62.

The guide plate 26 is a member that guides the cartridge 1 when the cartridge 1 is mounted on the mounting section 62 of the PCR device 100. A guiderail 63A is formed in the mounting section 62 of the PCR device 100. While the guide plate 26 of the tube 20 is guided along the guide rail 63A, the cartridge 1 is inserted into and fixed in the mounting section 62. The cartridge 1 has a long shape. However, since the cartridge 1 is inserted into the mounting section 62 while being guided by the guide plate 26, it is easy to fix the cartridge 1 in the normal position with respect to the mounting section 62.

The fixing claw 25 and the guide plate 26 are tabular members projecting from the left and right of the capillary 23. When the magnetic beads 7 in the tube 20 are moved by a magnet, the magnet is brought close to the magnetic beads 7 from a direction perpendicular to the fixing claw 25 and the guide plate 26 having the tabular shape. Consequently, it is possible to reduce the distance between the magnet and the magnetic beads 7 in the tube 20. However, the fixing claw 25 and the guide plate 26 may have other shapes as long as the distance between the magnet and the magnetic beads 7 in the tube 20 can be reduced.

### (2-3) PCR Container

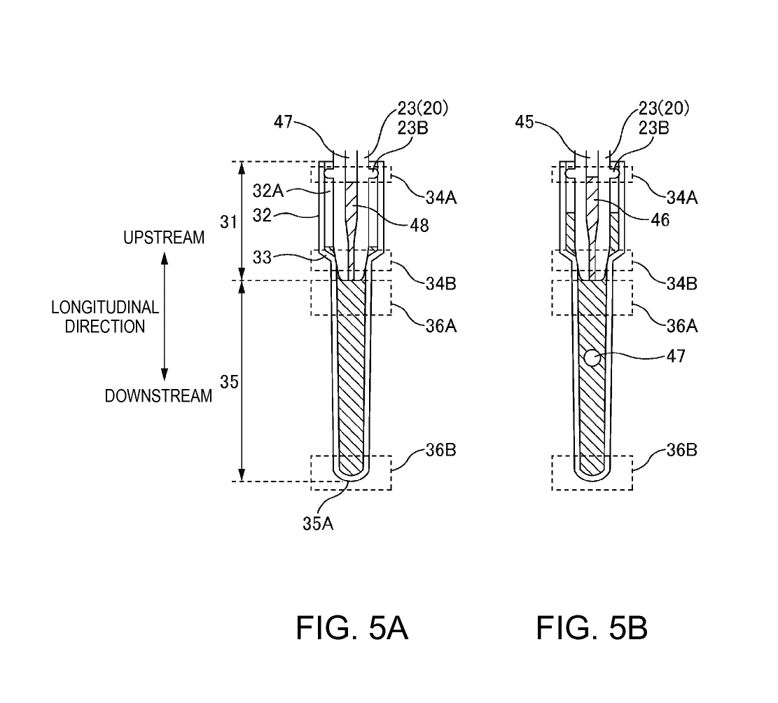

FIGS. 5A and 5B are explanatory diagrams of the periphery of the PCR container 30. FIG. 5A is an explanatory diagram of an initial state. FIG. 5B is an explanatory diagram of a state after the plunger 10 is pressed. The PCR container 30 is explained with reference to FIGS. 2A to 2C as well.

The PCR container 30 is a container that receives liquid pushed out from the tube 20 and is a container that stores the reaction solution droplet 47 during the thermal cycle treatment.

The PCR container 30 includes a seal forming section 31 and a channel forming section 35. The seal forming section 31 is a part in which the tube 20 is inserted and is a part that suppresses oil, which overflows from the channel forming section 35, from leaking to the outside. The channel forming section 35 is a part further on the downstream side than the seal forming section 31 and is a part that forms a channel through which the reaction solution droplet 47 moves. The PCR container 30 is fixed to the tube 20 in two places of an upper seal section 34A and a lower seal section 34B of the seal forming section 31.

The seal forming section 31 includes an oil receiving section 32 and a step section 33.

The oil receiving section 32 is a cylindrical part and functions as a reservoir that receives oil overflowing from the channel forming section 35. A gap is formed between the inner wall of the oil receiving section 32 and the outer wall of the capillary 23 of the tube 20. The gap is an oil receiving space 32A that receives the oil overflowing from the channel forming section 35. The volume of the oil receiving space 32A is larger than a volume of the slide of the seal 12A of the plunger 10 in the lower syringe 22 of the tube 20.

The inner wall on the upstream side of the oil receiving section 32 comes into contact with the annular convex section of the tube 20, whereby the upper seal section 34A is formed. The upper seal section 34A is a seal that suppresses, while allowing passage of the air, the oil in the oil receiving space 32A from leaking to the outside. In the upper seal section 34A, a vent hole is formed to a degree in which the oil does not leak with the surface tension of the oil. The vent hole of the upper seal section 34A may be a gap between the convex section of the tube 20 and the inner wall of the oil receiving section 32 or may be a hole, a groove, or a cutout formed in the convex section of the tube 20. The upper seal section 34A may be formed by an oil absorbing material that absorbs the oil.

The step section 33 is a part with a step provided on the downstream side of the oil receiving section 32. The inner diameter of a downstream section of the step section 33 is smaller than the inner diameter of the oil receiving section 32. The inner wall of the step section 33 is in contact with the outer wall on the downstream side of the capillary 23 of the tube 20. The inner wall of the step section 33 and the outer wall of the tube 20 come into contact with each other, whereby the lower seal section 34B is formed. The lower seal section 34B is a seal that, while allowing the oil in the channel forming section 35 to flow to the oil receiving space 32A, resists the flow of the oil. Because of a pressure loss in the lower seal section 34B, the pressure in the channel forming section 35 is higher than the outside pressure. Therefore, even if the liquid in the channel forming section is heated during the thermal cycle treatment, air bubbles less easily occur in the liquid in the channel forming section 35.

The channel forming section 35 is an annular member and is a container that forms a channel through which the reaction solution droplet 47 moves. Oil is filled in the channel forming section 35. The upstream side of the channel forming section 35 is closed by the terminal end of the tub 20. The terminal end of the tube 20 is opened toward the channel forming section 35. The inner diameter of the channel forming section 35 is larger than the inner diameter of the capillary 23 of the tube 20 and is larger than the outer diameter of the liquid formed in a spherical shape equivalent to the capacity of the reaction solution plug 47. The inner wall of the channel forming section 35 desirably has water repellency to a degree in which a water-soluble reaction solution does not adhere to the inner wall.

Note that the upstream side of the channel forming section 35 is heated to a relatively high temperature (e.g., about 95° C.) by a high-temperature side heater 65B on the outside and forms a high-temperature region 36A. The downstream side of the channel forming section 35 is heated to a relatively low temperature (e.g., about 60° C.) by a low-temperature side heater 65C on the outside and forms a low-temperature region 36B. The bottom 35A (an end on the downstream side) of the PCR container 30 is included in the low-temperature region 36B. Consequently, a temperature gradient is formed in the liquid in the channel forming section 35.

As shown in FIG. 5A, in the initial state, the oil is filled in the channel forming section 35 of the PCR container 30. The interface of the oil is located relatively on the downstream side of the oil receiving space 32A. The volume further on the upstream side than the interface of the oil in the oil receiving space 32A is larger than a volume of the slide of the seal 12A of the plunger 10 in the lower syringe 22 of the tube 20.

As shown in FIG. 5B, when the plunger 10 is pushed, the liquid in the tube 20 is pushed out to the channel forming section 35. Oil is filled in the channel forming section 35 in advance. The liquid in the tube 20 is pushed out into the oil. Therefore, gas does not flow into the channel forming section 35.

When the plunger 10 is pushed, first, the third oil plug 48 in the tube 20 flows into the channel forming section 35. The oil equivalent to the third oil plug 48 flown into the channel forming section 35 flows into the oil receiving space 32A from the channel forming section 35. The oil interface of the oil receiving space 32A rises. At this point, the pressure of the liquid in the channel forming section 35 rises because of a pressure loss in the lower seal section 34B. After the third oil plug 48 is pushed out from the tube 20, the reaction solution plug 47 flows into the channel forming section 35 from the tube 20. Since the inner diameter of the channel forming section 35 is larger than the inner diameter of the capillary 23, the reaction solution plug 47 having a plug shape (a columnar shape) in the tube 20 changes to a droplet shape in the oil in the channel forming section 35. Note that the volume further on the upstream side than the interface of the oil in the initial state in the oil receiving space 32A is larger than the volume of the slide of the seal 12A of the plunger 10 in the lower syringe 22 of the tube 20. Therefore, the oil does not overflow from the oil receiving space 32A.

### Specific Gravity of Oil

It is preferable that at least one of the oils in the tube 20 such as the first to third oils and the fourth oil have different specific gravities. It is more preferable that all the oils in the tube 20 and the fourth oil have different specific gravities. In particular, it is preferable that the fourth oil has smaller specific gravity than at least one of the oils in the tube 20 such as the first to third oils. It is more preferable that the fourth oil has smaller specific gravity than all the oils in the tube 20. Note that, in this specification, reference substance of specific gravity is water of 4° C.

As explained above, as the plugs in the cartridge, the plugs formed by the water solutions such as the cleaning solution and the reaction solution and the plugs formed by the oils are alternately arranged to prevent the water solutions and the oils from being mixed with each other. However, since it is likely that the cartridge is shocked during use, storage, transportation, and the like, the plugs in the cartridge preferably have higher shock resistance. As explained in examples, in the cartridge, the shock resistance was larger when a specific gravity difference between the water solution plug and the oil plug was smaller. This is considered because, when a shock is received from the outside, if the specific gravity difference between the water solution plug and the oil plug is large, since a larger force is applied to the interface of the plugs, the interface tends to be disturbed.

In this case, concerning the oil in the tube 20 and the water solution in the tube 20 adjacent to each other, when the specific gravity of the oil is represented as Ap and the specific gravity of the water solution is represented as Bq, it is preferable to set Ap and Bq such that |Bq−Ap|≦j (j is a constant) holds. In |Bq−Aq|≦j, j is a constant that depends on, for example, the shape of the tube and is, for example, preferably 0.2, more preferably 0.15, and still more preferably 0.1. However, in order to keep centrifugal force resistance to be equal to or smaller than 64 G using a tube having an inner diameter of 1.0 mm, it is preferable to set j to 0.12 (see Experimental Example 3).

On the other hand, the reaction solution droplet 47 of the PCR solution is present in the oil in the PCR container 30. As explained below, during a thermal cycle, reversal of the PCR container 30 is repeated. According to the repetition of the reversal of the PCR container 30, the droplet 47 in the oil repeats movement of shifting to an upper part of the oil according to the reversal and sinking in the oil with a specific gravity difference between the droplet 47 and the oil. As explained in the experimental examples, moving speed was successfully increased by increasing a specific gravity difference between the PCR solution and the oil. This is considered to lead to a reduction in time required for the thermal cycle.

In this case, when the specific gravity of the oil in the PCR container 30 is represented as A₄ and the specific gravity of the reaction solution droplet 47 in the PCR container 30 is represented as Bq, A₄ and Bq need to be set such that Bq−A₄|≧k (k is a constant) holds. In |Bq−A₄|≧k, k is a constant that depends on, for example, the shape of the PCR container 30 and is, for example, preferably 0.1, more preferably 0.03, and still more preferably 0.1. However, in order to set the volume of the reaction solution droplet 47 to 1 μl and keep a moving time of the droplet to be equal to or smaller than 2 seconds using the PCR container 30 having an inner diameter of 2.0 mm and length of 25.0 mm, it is preferable to set k to 0.06 (see Experimental Example 3).

Note that, specifically, the specific gravity of the first to third oils is preferably equal to or larger than 0.88 and equal to or smaller than 1.10. The specific gravity of the fourth oil is preferably equal to or larger than 0.80 and equal to or smaller than 0.95. The specific gravity of the cleaning solution and the eluate is preferably equal to or larger than 1.00 and equal to or smaller than 1.20. However, the specific gravities are not particularly limited.

### PCR Device 100

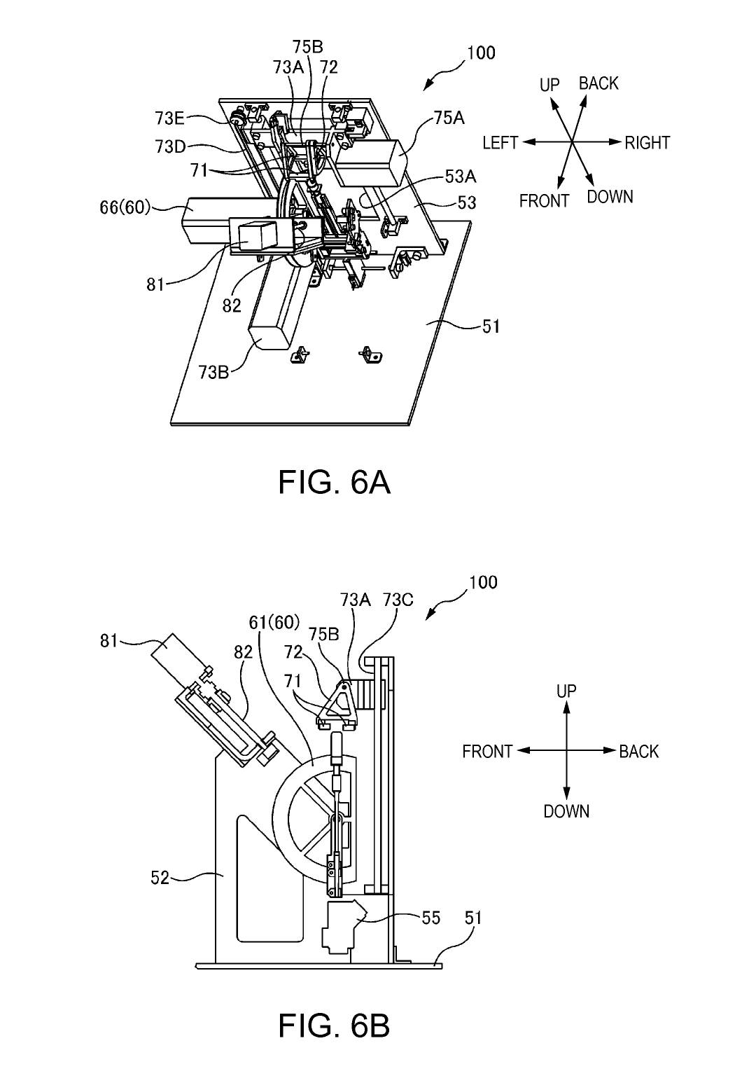

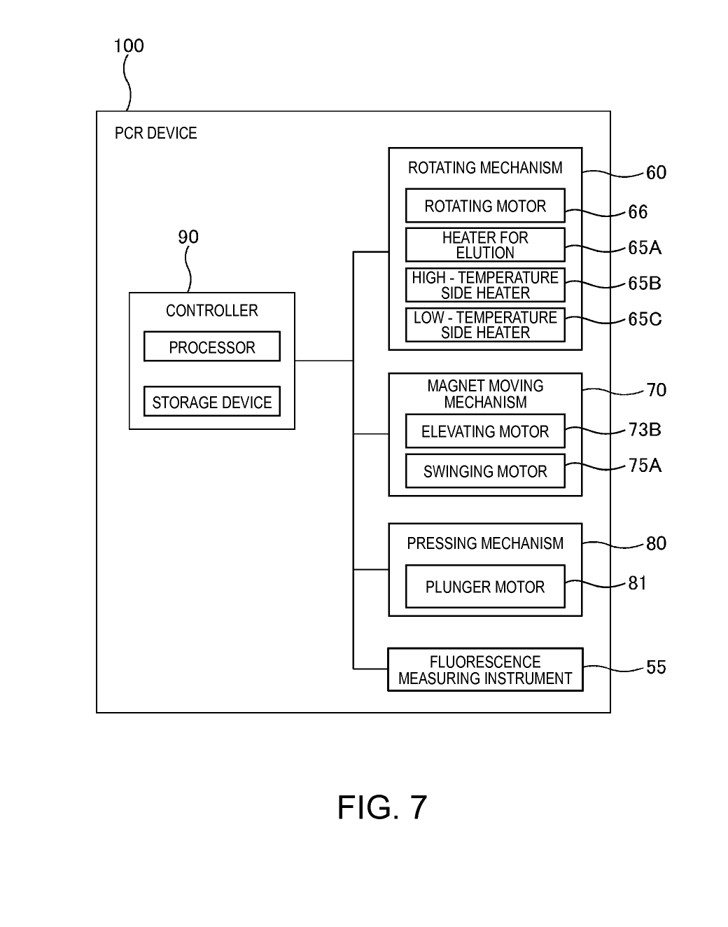

FIG. 6A is a perspective view of the internal configuration of the PCR device 100. FIG. 6B is a side view of a main configuration of the PCR device 100. FIG. 7 is a block diagram of the PCR device 100. The PCR device 100 performs the nucleic acid elution treatment and the thermal cycle treatment using the cartridge 1.

In the following explanation of the PCR device 100, up and down, front and back, and left and right are defined as shown in the figures. That is, the vertical direction at the time when a base 51 of the PCR device 100 is horizontally set is represented as “up-down direction”. “Up” and “down” are defined according to the gravity direction. The axis direction of the rotation axis of the cartridge 1 is represented as “left-right direction”. A direction perpendicular to the up-down direction and the left-right direction is represented as “front-back direction”. The side of a cartridge insertion port 53A viewed from the rotation axis of the cartridge 1 is represented as “back” and the opposite side is represented as “front”. The right side in the left-right direction viewed from the front side is represented as “right” and the left side is represented as “left”.

The PCR device 100 includes a rotating mechanism 60, a magnet moving mechanism 70, a pressing mechanism 80, a fluorescence measuring instrument 55, and a controller 90.

### (1) Rotating Mechanism 60

The rotating mechanism 60 is a mechanism for rotating the cartridge 1 and heaters. The rotating mechanism 60 vertically reverses the cartridge 1 and the heaters, whereby the reaction solution droplet 47 moves in the channel forming section 35 of the PCR container 30 and the thermal cycle treatment is performed.

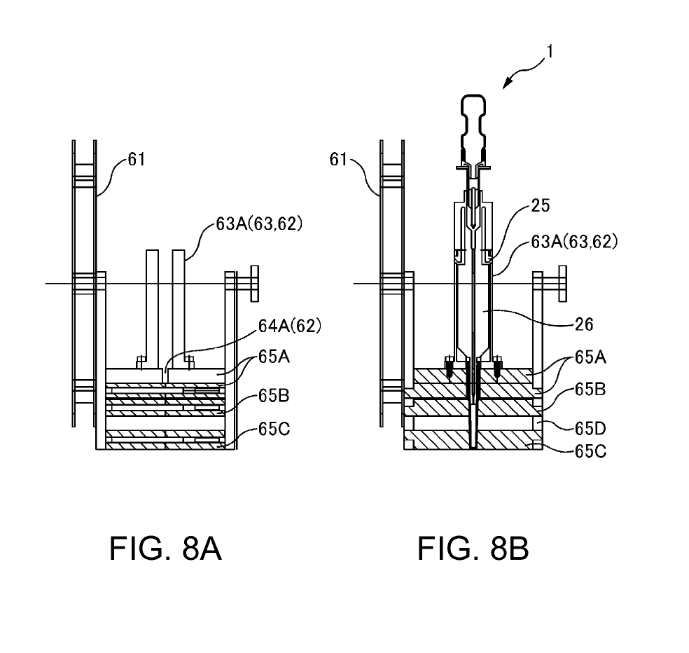

The rotating mechanism 60 includes a rotating body 61 and a rotating motor 66. FIG. 8A is an explanatory diagram of the rotating body 61. FIG. 8B is an explanatory diagram of a state in which the cartridge 1 is mounted on the mounting section 62 of the rotating body 61.

The rotating body 61 is a member rotatable about a rotating shaft. The rotating shaft of the rotating body 61 is supported by a supporting stand 52 fixed to the base 51. In the rotating body 61, the mounting section 62 on which the cartridge 1 is mounted and the heaters (a heater for elution 65A, a high-temperature side heater 65B, and a low-temperature side heater 65C) are provided. When the rotating body 61 rotates, it is possible to vertically reverse the cartridge 1 while maintaining a positional relation between the cartridge 1 and the heaters. The rotating motor 66 is a power source for rotating the rotating body 61. The rotating motor 66 rotates the rotating body 61 to a predetermined position according to an instruction from the controller 90. A transmission mechanism such as a gear may be interposed between the rotating motor 66 and the rotating body 61.

The mounting section 62 is a part on which the cartridge 1 is mounted. The mounting section 62 includes a fixing section 63 in which a notch is formed. An insertion hole 64A formed in the heaters (the heater for elution 65A, the high-temperature side heater 65B, and the low-temperature side heater 65C) also functions as the mounting section 62. The fixing claw 25 of the cartridge 1 is caught by the notch of the fixing section 63 in a state in which the PCR container 30 is inserted into the insertion hole 64A, whereby the cartridge 1 is mounted on the rotating body 61 (see FIGS. 4A and 4B). Apart of the heaters also functions as the mounting section 62. However, the mounting section 62 and the heaters may be separate. The mounting section 62 is indirectly fixed to the rotating body 61 via the heater for elution 65A. However, the mounting section 62 may be directly provided in the rotating body 61. The cartridge 1 mountable on the mounting section 62 is not limited to one cartridge. A plurality of the cartridges 1 may be mounted on the mounting section 62.

In the fixing section 63, the guiderail 63A is formed along the up-down direction (see FIGS. 4A and 4B). The guiderail 63A guides the guide plate 26 of the cartridge 1 in an inserting direction while restraining the guide plate 26 in the front-back direction. The cartridge 1 is inserted into the mounting section 62 while the guide plate 26 is guided by the guiderail 63A. Therefore, the PCR container 30 of the cartridge 1 is guided to the insertion hole 64A. The cartridge 1 is fixed in the normal position with respect to the mounting section 62.

The PCR device 100 includes the heater for elution 65A and includes the high-temperature side heater 65B and the low-temperature side heater 65C as heaters for PCR. Each of the heaters is configured by a heat generation source and a heat block not shown in the figure. The heat generation source is, for example, a cartridge heater and is inserted into the heat block. The heat block is metal such as aluminum having high thermal conductivity. The heat block suppresses heat unevenness and heats the liquid in the cartridge 1 with heat from the heat generation source. The heat block is desirably a nonmagnetic body such that magnets 71 for moving the magnetic beads 7 do not attract the heat block.

The heater for elution 65A is a heater that heats the fourth plug 47 of the cartridge 1. When the cartridge 1 is fixed in the normal position, the heater for elution 65A is opposed to the fourth plug 47 of the tube 20. For example, the heater for elution 65A heats the fourth plug 47 to about 50° C., whereby separation of nucleic acid from magnetic beads is facilitated.

The high-temperature side heater 65B is a heater that heats the upstream side of the channel forming section 35 of the PCR container 30. When the cartridge 1 is fixed in the normal position, the high-temperature side heater 65B is opposed to the upstream side (the high-temperature region 36A) of the channel forming section 35 of the PCR container 30. For example, the high-temperature side heater 65B heats liquid on the upstream side of the channel forming section 35 of the PCR container 30 to about 90 to 100° C.

The low-temperature side heater 65C is a heater that heats the bottom 35A of the channel forming section 35 of the PCR container 30. When the cartridge 1 is fixed in the normal position, the low-temperature side heater 65C is opposed to the downstream side (the low temperature region 36B) of the channel forming section 35 of the PCR container 30. For example, the low-temperature side heater 65C heats liquid in the low-temperature region 36B of the PCR container 30 to about 50 to 75° C.

A spacer 65D is arranged between the high-temperature side heater 65B and the low-temperature side heater 65C. The spacer 65D suppresses heat conduction between the high-temperature side heater 65B and the low-temperature side heater 65C. The spacer 65D is also used for accurately setting the distance between the high-temperature side heater 65B and the low-temperature side heater 65C. Consequently, a temperature gradient is formed in the liquid in the channel forming section 35 of the PCR container 30 by the high-temperature side heater 65B and the low-temperature side heater 65C.

In the heat blocks respectively forming the heater for elution 65A, the high-temperature side heater 65B, and the low-temperature side heater 65C, through-holes forming the through-hole 64A are respectively formed. The outer wall of the bottom 35A of the PCR container 30 is exposed from a lower side opening of the insertion hole 64A of the low-temperature side heater 65C. The fluorescence measuring instrument 55 measures the luminance of the reaction solution droplet 47 from the opening on the lower side of the insertion hole 64A.

Note that temperature control devices are respectively provided in the high-temperature side heater 65B and the low-temperature side heater 65C. The high-temperature side heater 65B and the low-temperature side heater 65C can be set to temperatures suitable for polymerase reactions of the heaters.

### (2) Magnet Moving Mechanism 70

The magnet moving mechanism 70 is a mechanism for moving the magnets 71. The magnet moving mechanism 70 attracts the magnetic beads 7 in the cartridge 1 to the magnets 71 and moves the magnets 71 to thereby move the magnetic beads 7 in the cartridge 1. The magnet moving mechanism 70 includes a pair of magnets 71, an elevating mechanism 73, and a swinging mechanism 75.

The magnets 71 are members that attract the magnetic beads 7. As the magnets 71, a permanent magnet, an electromagnet, or the like can be used. In this embodiment, the permanent magnet that does not cause heat generation and the like is used. The pair of magnets 71 is retained by an arm 72 to set the positions of the magnets 71 in the up-down direction substantially the same such that the magnets 71 are opposed to each other in the front-back direction. The magnets 71 can be opposed to each other from the front side or the back side of the cartridge 1 mounted on the mounting section 62. The pair of magnets 71 can hold the cartridge 1, which is mounted on the mounting section 62, from the front-back direction. The magnets 71 are opposed to each other from a direction (the front-back direction) orthogonal to a direction (the left-right direction) in which the fixing claw 25 and the guide plate 26 of the cartridge 1 are provided, whereby the distance between the magnetic beads 7 in the cartridge 1 and the magnets 71 can be reduced.

The elevating mechanism 73 is a mechanism for moving the magnets 71 in the up-down direction. Since the magnets 71 attract the magnetic beads 7, if the magnets 71 are moved in the up-down direction according to the movement of the magnetic beads 7, it is possible to induce the magnetic beads 7 in the cartridge 1 in the up-down direction.

The elevating mechanism 73 includes a carriage 73A that moves in the up-down direction and an elevating motor 73B. The carriage 73A is a member movable in the up-down direction and is guided to be movable in the up-down direction by a carriage guide 73C provided on a sidewall 53 where the cartridge insertion port 53A is present. The arm 72 for retaining the pair of magnets 71 is attached to the carriage 73A. Therefore, when the carriage 73A moves in the up-down direction, the magnets 71 move in the up-down direction. The elevating motor 73B is a power source for moving the carriage 73A in the up-down direction. The elevating motor 73B moves the carriage 73A to a predetermined position in the up-down direction according to an instruction from the controller 90. The elevating motor 73B moves the carriage 73A in the up-down direction using a belt 73D and a pulley 73E. However, the elevating motor 73B may move the carriage 73A in the up-down direction using other transmission mechanisms.

When the carriage 73A is present in the top position (a retracted position), the magnets 71 are located further on the upper side than the cartridge 1. When the carriage 73A is present in the retracted position, even if the cartridge 1 rotates, the elevating mechanism 73 does not come into contact with the cartridge 1. The elevating mechanism 73 can lower the position of the carriage 73A to a position where the magnets 71 are opposed to the reaction solution plug 47. Consequently, the elevating mechanism 73 can move the magnets 71 to move the magnetic beads 7 in the tank 3 to the position of the reaction solution plug 47.

The swinging mechanism 75 is a mechanism for swinging the pair of magnets 71 in the front-back direction. When the pair of magnets 71 is swung in the front-back direction, the intervals between the magnets 71 and the cartridge 1 alternately change. Since the magnetic beads 7 are attracted to the magnet 71 present at a shorter distance, the pair of magnets 71 is swung in the front-back direction, whereby the magnetic beads 7 in the cartridge 1 move in the front-back direction.

The swinging mechanism 75 includes a swinging motor 75A and a gear. The swinging motor 75A and the gear are provided in the carriage 73A and are movable in the up-down direction together with the carriage 73A. The power of the swinging motor 75A is transmitted to the arm 72 via the gear, whereby the arm 72 for retaining the magnets 71 rotates about a swing rotating shaft 75B with respect to the carriage 73A. In order to prevent the magnets 71 from coming into contact with the cartridge 1 to damage the cartridge 1, the swinging mechanism 75 swings the magnets 71 in a range in which the magnets 71 and the cartridge 1 are not in contact with each other.

The swing rotating shaft 75B is a rotating shaft of the arm 72. The swing rotating shaft 75B is parallel to the left-right direction to be capable of swinging the magnets 71 in the front-back direction. When the swing rotating shaft 75B is viewed from the right or the left, the swing rotating shaft 75B is arranged to be shifted further to the front side or the back side than the cartridge 1. Consequently, when the carriage 73A moves downward, it is possible to prevent contact of the cartridge 1 and the arm 72. Note that the swing rotating shaft 75B may be a shaft parallel to the up-down direction as long as the magnets 71 can be swung in the front-back direction.

### (3) Pressing Mechanism 80

The pressing mechanism 80 is a mechanism for pressing the plunger 10 of the cartridge 1. The plunger 10 is pressed by the pressing mechanism 80, whereby the reaction solution plug 47 and the oil plug 48 of the cartridge 1 are pushed out to the PCR container 30 and the reaction solution droplet 47 is formed in the oil in the PCR container 30.

The pressing mechanism 80 includes a plunger motor 81 and a rod 82. The plunger motor 81 is a power source that moves the rod 82. The rod 82 is a member that presses the attachment stand 11A of the plunger 10 of the cartridge 1. The attachment stand 11A is pushed rather than the tank 3 of the cartridge 1 because the tank 3 is expandably formed of resin having flexibility. When the tank 3 is not deformed, the pressing mechanism 80 may push the tank 3 to thereby push the plunger 10.

A direction in which the rod 82 pushes the plunger 10 is not the up-down direction and tilted 45 degrees with respect to the up-down direction. Therefore, when the plunger 10 is pushed by the pressing mechanism 80, the PCR device 100 rotates the rotating body 61 45 degrees and adjusts the longitudinal direction of the cartridge 1 to the moving direction of the rod 82 and then moves the rod 82. Since the direction in which the rod 82 pushes the plunger 10 is tilted 45 degrees with respect to the up-down direction, it is easy to arrange the pressing mechanism 80 not to interfere with the elevating mechanism 73. Since the direction in which the rod 82 pushes the plunger 10 is tilted 45 degrees with respect to the up-down direction, it is possible to reduce the dimension in the up-down direction of the PCR device 100.

### (4) Fluorescence Measuring Instrument 55

The fluorescence measuring instrument 55 is a measuring instrument that measures the luminance of the reaction solution droplet 47 of the PCR container 30. The fluorescence measuring instrument 55 is arranged further on the lower side than the rotating body 61 to be opposed to the bottom 35A of the PCR container 30 of the cartridge 1. The fluorescence measuring instrument 55 measures the luminance of the reaction solution droplet 47 present on the bottom 35A of the PCR container 30 from the lower side opening of the insertion hole 64A of the low-temperature side heater 65C.

### (5) Controller 90

The controller 90 is a control section that performs control of the PCR device 100. The controller 90 includes a processor such as a CPU and a storage device such as a ROM or a RAM. In the storage device, various computer programs and data are stored. The storage device provides an area in which the computer programs are expanded. The processor executes the computer programs stored in the storage device, whereby various kinds of processing are realized.

For example, the controller 90 controls the rotating motor 66 to rotate the rotating body 61 to a predetermined rotating position. A not-shown rotating position sensor is provided in the rotating mechanism 60. The controller 90 drives and stops the rotating motor 66 according to a detection result of the rotating position sensor.

The controller 90 controls the heaters (the heater for elution 65A, the high-temperature side heater 65B, and the low-temperature side heater 65C) to generate heat. Not-shown temperature sensors are provided in the heat blocks included in the heaters. The controller 90 controls the cartridge heaters to turn on and off according to detection results of the temperature sensors.

The controller 90 controls the elevating motor 73B to move the magnets 71 in the up-down direction. A not-shown position sensor for detecting the position of the carriage 73A is provided in the PCR device 100. The controller 90 drives and stops the elevating motor 73B according to a detection result of the position sensor.

The controller 90 controls the swinging motor 75A to swing the magnets 71 in the front-back direction. A position sensor for detecting the position of the arm 72 for retaining the magnets 71 is provided in the PCR device 100. The controller 90 drives and stops the swinging motor 75A according to a detection result of the position sensor.

The controller 90 controls the fluorescence measuring instrument 55 to measure the luminance of the reaction solution droplet 47 of the PCR container 30. The controller 90 causes the fluorescence measuring instrument 55 to perform measurement when the fluorescence measuring instrument 55 is opposed to the bottom 35A of the PCR container 30 of the cartridge 1. A measurement result is stored in the storage device.

### Operation Explanation

### (1) Mounting Operation for the Cartridge 1

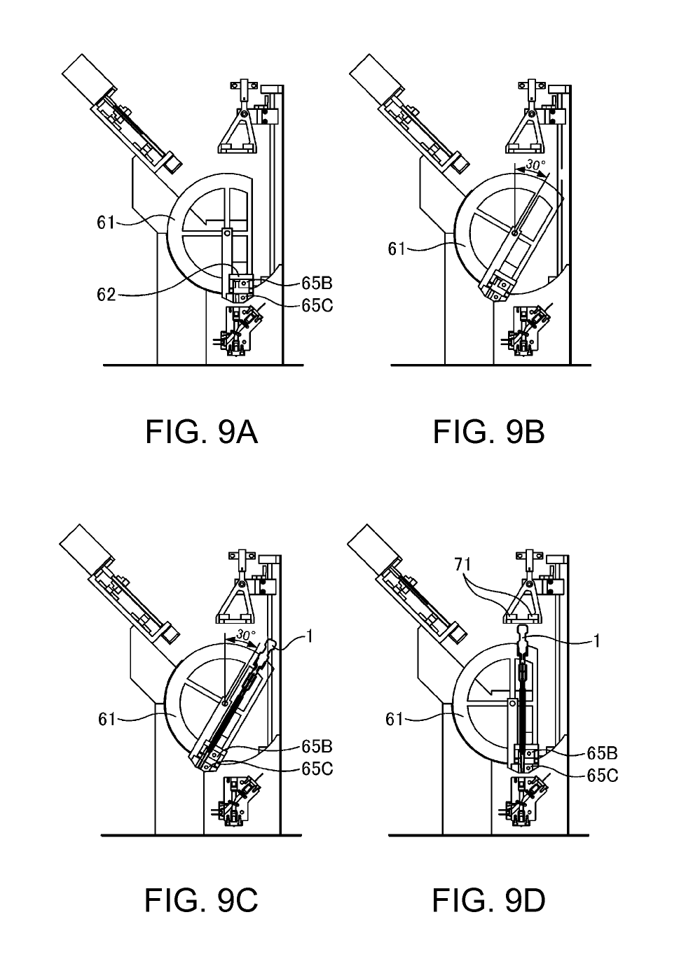

FIGS. 9A to 9D are explanatory diagrams of states of the PCR device 100 during the mounting of the cartridge 1. FIG. 9A is an explanatory diagram of an initial state before the mounting of the cartridge 1. FIG. 9B is an explanatory diagram of a standby state. FIG. 9C is an explanatory diagram immediately after the mounting of the cartridge 1. FIG. 9D is an explanatory diagram of an initial state in a mounted state of the cartridge 1.

As shown in FIG. 9A, in the initial state before the mounting of the cartridge 1, a mounting direction of the mounting section 62 is the up-down direction. In the following explanation, a rotating position of the rotating body 61 is indicated with a rotating position of the rotating body 61 in this state set as a reference (0 degree) and the counterclockwise direction viewed from the right set as a positive direction.

As shown in FIG. 9B, the controller 90 drives the rotating motor 66 to rotate the rotating body 61 −30 degrees. In this state, the operator inserts the cartridge 1 into the mounting section 62 from the cartridge insertion port 53A. In this case, the cartridge 1 is inserted into the mounting section 62 while the guide plate 26 is guided by the guiderail 63A. Therefore, the PCR container 30 of the cartridge 1 is guided to the insertion hole 64A of the mounting section 62. The operator inserts the cartridge 1 until the fixing claw 25 of the cartridge 1 is caught by the notch of the fixing section 63. Consequently, the cartridge 1 is fixed in a normal position with respect to the mounting section 62. If the PCR container 30 is not inserted into the insertion hole 64A and the cartridge 1 is present in an abnormal position with respect to the mounting section 62, the fixing claw 25 of the cartridge 1 is not caught by the notch of the fixing section 63. Therefore, the operator can recognize that the cartridge 1 is present in the abnormal position.

As shown in FIG. 9C, when the cartridge 1 is fixed in the normal position with respect to the mounting section 62, the reaction solution plug 47 of the tube 20 is opposed to the heater for elution 65A, the upstream side (the high-temperature region 36A) of the channel forming section 35 of the PCR container 30 is opposed to the high-temperature side heater 65B, and the downstream side (the low-temperature region 36B) of the channel forming section 35 of the PCR container 30 is opposed to the low-temperature side heater 65C. Since the mounting section 62 and the heaters are provided in the rotating body 61, even if the rotating body 61 rotates, the positional relation between the cartridge 1 and the heaters is maintained.

After the cartridge 1 is mounted on the mounting section 62, as shown in FIG. 9D, the controller 90 rotates the rotating body 61 30 degrees and returns the position of the rotating body 61 to the reference. Note that the controller 90 may detect the mounting of the cartridge 1 on the mounting section 62 using a not-shown sensor or may detect the mounting according to an input operation from the operator.

### (2) Nucleic Acid Elution Treatment

### Up Down Movement of the Magnets 71

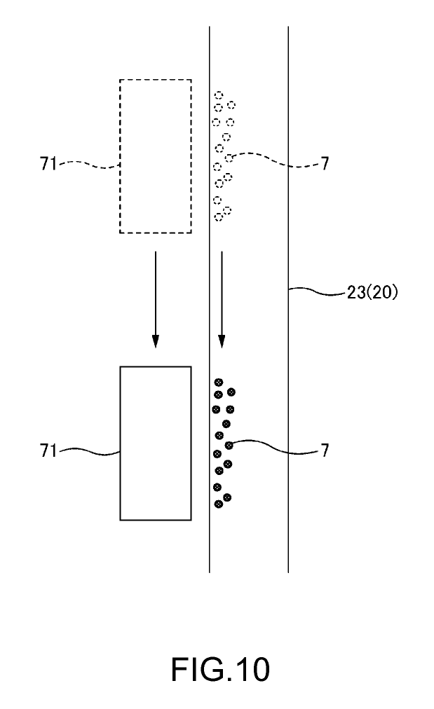

FIG. 10 is a conceptual diagram of the behavior of the magnetic beads 7 at the time when the magnets 71 are moved in the downward direction. The magnetic beads 7 in the cartridge 1 are attracted to the magnets 71. Therefore, when the magnets 71 move outside the cartridge 1, the magnetic beads 7 in the cartridge 1 move together with the magnets 71.

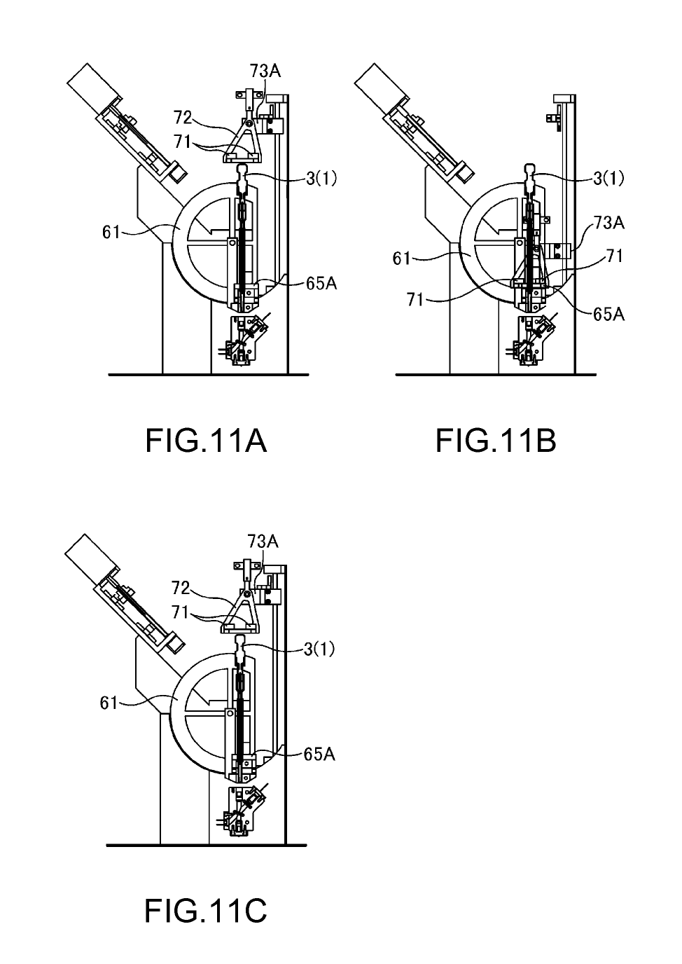

FIGS. 11A to 11C are explanatory diagrams of nucleic acid elution treatment. FIG. 11A is an explanatory diagram of a state of the PCR device 100 before the nucleic acid elution treatment. FIG. 11B is an explanatory diagram of a state of the PCR device 100 at the time when the magnets 71 are moved to the reaction solution plug 47. FIG. 11C is an explanatory diagram of a state of the PCR device 100 at the time when the magnets 71 are lifted.

As shown in FIG. 11A, the longitudinal direction of the cartridge 1 in the initial state is parallel to the vertical direction with the tank 3 facing up. In this state, as shown in FIG. 2A, the cartridge 1 includes, in order from the top, the solution 41 (the tank 3) including the magnetic beads 7, the oil 42 (the plunger 10), the cleaning solution 43 (the upstream side of the tube 20), the first oil plug 44 (the capillary 23), the cleaning solution plug 45 (the capillary 23), the second oil plug 46 (the capillary 23), the reaction solution plug 47 (the capillary 23), the third oil plug 48 (the capillary 23), and the oil (the PCR container 30).

As shown in FIG. 11A, in the initial state, the carriage 73A is present in the top position (the retracted position). The magnets 71 are located further on the upper side than the cartridge 1. In this state, the controller 90 drives the elevating motor 73B to gradually move the carriage 73A in the downward direction and gradually move the magnets in the downward direction. Note that, since the longitudinal direction of the cartridge 1 is parallel to the vertical direction, the magnets 71 move along the cartridge 1.

When the magnets 71 move in the downward direction, the magnets 71 are opposed to the tank 3. The magnetic beads 7 in the tank 3 are attracted to the magnets 71. The controller 90 moves the carriage 73A in the downward direction at speed of a degree in which the magnetic beads 7 can move together with the magnets 71.

When the magnets 71 move from a position opposed to the tank 3 (the height of the tank 3) to a position opposed to the plunger 10 (the height of the plunger 10), the magnetic beads 7 pass through the opening on the upstream side of the cylindrical section 11 of the plunger 10 and pass the interface between the solution 41 in the tank 3 and the oil 42 on the upstream side of the cartridge main body 9. Consequently, the magnetic beads 7 bound with nucleic acid are introduced into the cartridge main body 9. When the magnetic beads 7 pass the interface with the oil 42, the solution 41 is wiped by the oil 42. Therefore, components of the solution 41 are less easily brought into the oil 42. Consequently, it is possible to suppress the components of the solution 41 from being mixed in the cleaning solution plug 45 and the reaction solution plug 47.

When the magnets 71 move in the downward direction in a state in which the magnets 71 are opposed to the plunger 10, the magnetic beads 7 pass through the inside of the cylindrical section 11, pass through the front and the back of the ribs 13, and exit from the opening on the downstream side of the cylindrical section 11 to be introduced into the upper syringe 21 of the tube 20. During this period, the magnetic beads 7 pass the interface between the oil 42 and the cleaning solution 43 in the plunger 10. When the magnetic beads 7 are introduced into the cleaning solution 43, the nucleic acid combining with the magnetic beads 7 is cleaned by the cleaning solution 43.

At this stage, the bar-like section 12 of the plunger 10 is not inserted into the lower syringe 22 of the tube 20. Therefore, when the magnets 71 move to a position opposed to the upper syringe 21 (the height of the upper syringe 21) to a position opposed to the capillary 23 (the height of the capillary 23), the magnetic beads 7 move from the upper syringe 21 to the lower syringe 22 and move from the lower syringe 22 to the capillary 23. The first oil plug 44 is present on the upstream side of the capillary 23. When the magnetic beads 7 move from the lower syringe 22 to the capillary 23, the magnetic beads 7 pass the interface between the cleaning solution 43 and the oil. In this case, since the cleaning solution 43 is wiped by the oil, components of the cleaning solution 43 are less easily brought into the oil. Consequently, it is possible to suppress the components of the cleaning solution 43 from being mixed in the cleaning solution plug 45 and the reaction solution plug 47.

When the magnets 71 move from a position opposed to the first oil plug 44 (the height of the first oil plug 44) to a position opposed to the cleaning solution plug 45 (the height of the cleaning solution plug 45), the magnetic beads 7 pass the interface between the oil and the cleaning solution. When the magnetic beads 7 are introduced into the cleaning solution plug 45, the nucleic acid bound to the magnetic beads 7 is cleaned by the cleaning solution.

When the magnets 71 move from the position opposed to the cleaning solution plug 45 (the height of the cleaning solution plug 45) to a position opposed to the second oil plug 46 (the height of the second oil plug 46), the magnetic beads 7 pass the interface between the cleaning solution and the oil. In this case, since the cleaning solution is wiped by the oil, the components of the cleaning solution are less easily brought into the oil. Consequently, it is possible to suppress the components of the cleaning solution from being mixed in the reaction solution plug 47.

When the magnets 71 move the position opposed to the second oil plug 46 (the height of the second oil plug 46) to a position opposed to the reaction solution plug 47 (the height of the reaction solution plug 47), the magnetic beads 7 pass the interface between the second oil plug 46 and the reaction solution plug 47.

Before the magnetic beads 7 are introduced into the reaction solution plug 47, the controller 90 controls the heater for elution 65A to heat the reaction solution plug 47 to about 50° C. Since the reaction solution plug 47 is heated before the magnetic beads 7 are introduced, it is possible to reduce time from the introduction of the magnetic beads 7 into the reaction solution plug 47 until elution of nucleic acid ends.

As shown in FIG. 11B, after the magnets 71 move to a position opposed to the reaction solution plug 47 (the height of the reaction solution plug 47), the controller 90 stops the elevating motor 73B to stop the movement of the magnets 71 in the up-down direction and treats the magnetic beads 7 for thirty seconds at 50° C. Then, the nucleic acid bound to the magnetic beads 7 is separated into the solution of the reaction solution plug 47 and reverse transcription reaction proceeds. By heating the reaction solution plug 47, the elution of the nucleic acid from the magnetic beads 7 and the reverse transcription reaction are facilitated.

After eluting the nucleic acid in the reaction solution plug 47, the controller 90 drives the elevating motor 73B in the opposite direction to gradually move the carriage 73A in the upward direction and gradually move the magnets 71 in the upward direction. The controller 90 moves the carriage 73A in the upward direction at speed of a degree in which the magnetic beads 7 can move together with the magnets 71.

When the magnets 71 move in the upward direction in the state shown in FIG. 11B, the magnetic beads 7 move from the reaction solution plug 47 to the second oil plug 46. The magnetic beads 7 are removed from the reaction solution plug 47.

When the magnets 71 gradually move to the position opposed to the upper syringe 21, the magnetic beads 7 also move to the upper syringe 21. The magnetic beads 7 are present further on the upper side than the lower syringe 22. If the magnetic beads 7 are moved to this position, the magnetic beads 7 are not introduced into the PCR container 30 when the plunger 10 is pushed. Therefore, in a period from this state to a state shown in FIG. 11C, the controller 90 may move the carriage 73A in the upward direction at speed of a degree in which the magnetic beads 7 cannot follow the movement of the magnets 71. Note that, if the magnetic beads 7 are not introduced into the PCR container 30 when the plunger 10 is pushed, the moving speed of the carriage 73A may be increased at an earlier stage.

Information concerning the moving speed of the magnets 71 is stored in the storage device of the controller 90. The controller 90 executes the operation explained above (the operation for moving the magnets 71 up and down) according to the information.

### Swing of the Magnets 71

While moving the magnets 71 in the up-down direction, the controller 90 may drive the swinging motor 75A to swing the pair of magnets 71, which holds the cartridge 1, in the front-back direction.

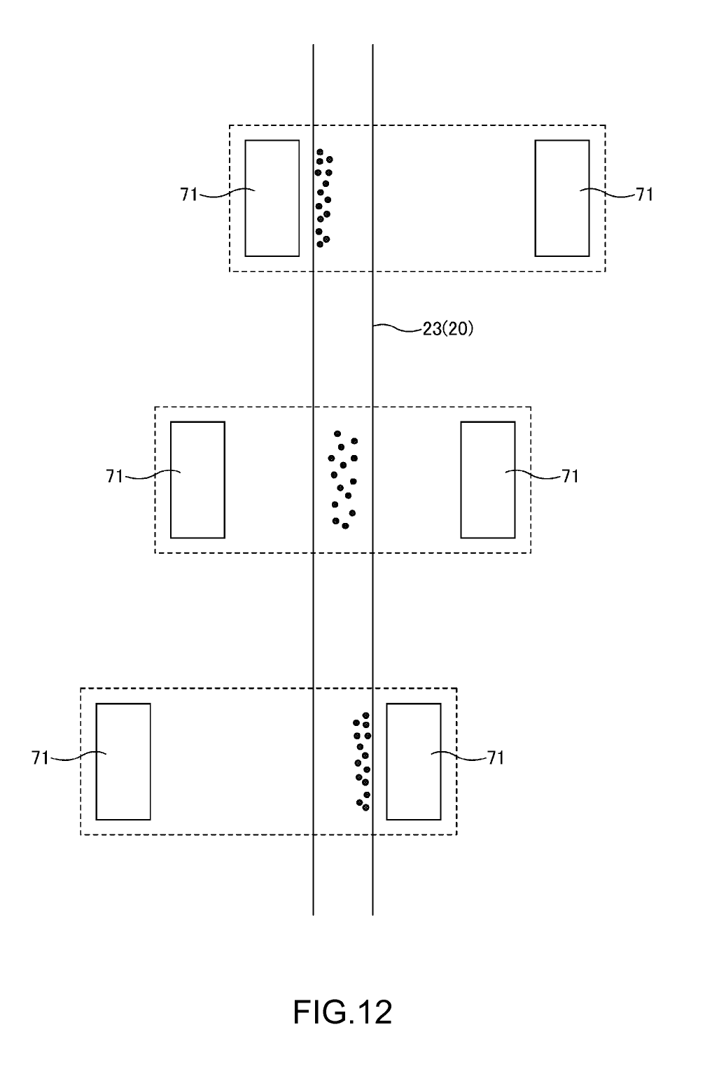

FIG. 12 is a conceptual diagram of the behavior of the magnetic beads 7 at the time when the magnets 71 are swung.

While the magnets 71 move in the up-down direction, the tube 20 is held between the pair of magnets 71 from the front-back direction. Since the pair of magnets 71 is retained by the arm 72, the distance in the front-back direction between the pair of magnets 71 is substantially fixed. Therefore, when one of the pair of magnets 71 approaches the tube 20, the other separates from the tube 20.

The magnetic beads 7 are attracted to the magnet 71 present at a shorter distance. Therefore, when one magnet 71 is close to the tube 20, the magnetic beads 7 are attracted to the side of the magnet 71. Thereafter, when the magnet 71 separates from the tube 20 and the magnet 71 on the opposite side approaches the tube 20, the magnetic beads 7 are attracted to the magnet 71 on the opposite side. Consequently, the magnetic beads 7 move in the front-back direction. When the pair of magnets 71 is swung in the front-back direction, the magnetic beads 7 reciprocate in the front-back direction.

When the magnetic beads 7 reciprocate in the front-back direction, liquid tends to come into contact with the magnetic beads 7. In particular, since the liquid in the capillary 23 hardly has fluidity, when it is desired to bring the liquid in the capillary 23 into contact with the magnetic beads 7 as much as possible, the reciprocation of the magnetic beads 7 in the front-back direction is effective.

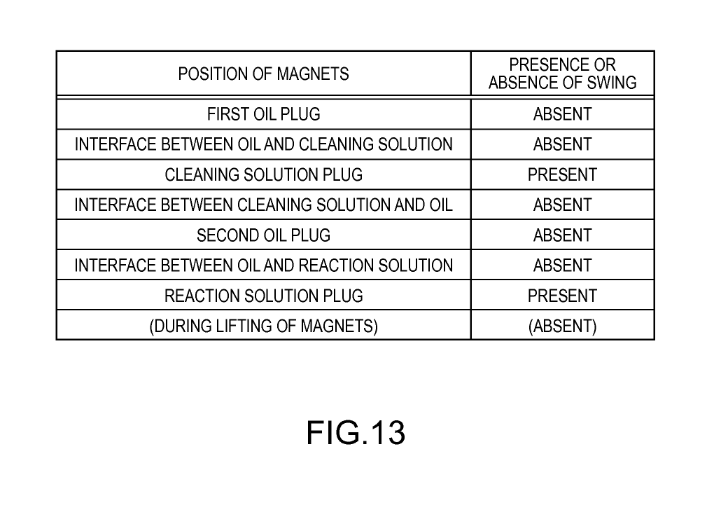

FIG. 13 is a table showing presence or absence of the swing of the magnets 71.

When the magnetic beads 7 move in the downward direction in the oil plug (the first oil plug 44 or the second oil plug 46), the controller 90 stops the swinging motor 75A not to swing the magnets 71. In this case, the controller 90 moves the magnets 71 in the downward direction in a state in which one of the pair of magnets 71 is brought close to the tube 20. This is because the magnetic beads 7 easily follow the movement of the magnets 71 compared with the case where the distances between the magnets 71 and the tube 20 are set equal.

When the magnetic beads 7 move in the downward direction in the cleaning solution plug 45, the controller 90 drives the swinging motor 75A to swing the magnets 71 in the front-back direction. Consequently, the magnetic beads 7 move in the downward direction while swinging in the front-back direction in the cleaning solution plug 45. Therefore, it is possible to improve cleaning efficiency of the magnetic beads 7. Further, since the cleaning efficiency is improved, it is possible to suppress an amount of the cleaning solution plug 45 and attain a reduction in the size of the cartridge 1.

When the magnetic beads 7 pass the interface between the cleaning solution and the oil (the second oil plug 46), the controller 90 stops the swinging motor 75A not to swing the magnets 71. Consequently, the magnetic beads 7 pass the interface without swinging. Therefore, the components of the cleaning solution are less easily brought into the oil. Note that the controller 90 moves the magnets 71 in the downward direction in a state in which one of the pair of magnets 71 is brought close to the tube 20. Consequently, the magnetic beads 7 are attracted to the magnet 71 present at a short distance and condense. The cleaning solution adhering to the magnetic beads 7 is squeezed. Therefore, the components of the cleaning solution are less easily brought into the oil.

When the magnetic beads 7 are present in the reaction solution plug 47, the controller 90 drives the swinging motor 75A to swing the magnets 71 in the front-back direction. Consequently, the magnetic beads 7 swing in the front-back direction in the reaction solution plug 47. Therefore, it is possible to improve elution efficiency of the nucleic acid bound to the magnetic beads 7. Since the elution efficiency is improved, it is possible to reduce time from the introduction of the magnetic beads 7 into the reaction solution plug 47 until the elution of the nucleic acid is ended.

Note that, after eluting the nucleic acid in the reaction solution plug 47, when moving the magnets 71 in the upward direction to lift the magnetic beads 7, the controller 90 stops the swinging motor 75A not to swing the magnets 71. In this case, the controller 90 moves the magnets 71 in the downward direction in a state in which one of the pair of magnets 71 is brought close to the tube 20. Consequently, the magnetic beads 7 easily follow the movement of the magnets 71. It is possible to increase the moving speed of the magnets 71.

In the storage device of the controller 90, information concerning the positions of the plugs of the capillary 23 and swing information shown in FIG. 13 are stored. The controller 90 executes the operation explained above (the operation for swinging the magnets 71) according to the information.

### (3) Droplet Forming Treatment

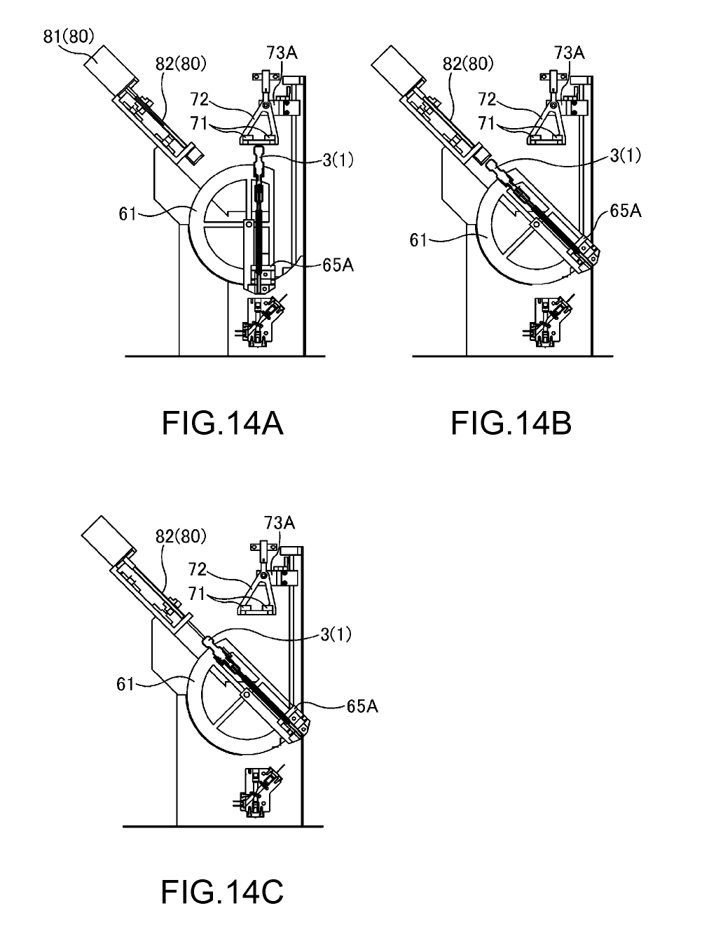

FIGS. 14A to 14C are explanatory diagrams of droplet forming treatment. FIG. 14A is an explanatory diagram of a state of the PCR device 100 at the time when the magnets 71 are lifted. FIG. 14B is an explanatory diagram of a state in which the rotating body 61 is rotated 45 degrees. FIG. 14C is an explanatory diagram of a state in which the rod 82 of the pressing mechanism 80 pushes the plunger 10.

As shown in FIG. 14A, when the carriage 73A is present in the retracted position, even if the cartridge 1 rotates, the elevating mechanism 73 does not come into contact with the cartridge 1. In such a state, the controller 90 rotates the rotating body 61 45 degrees.

As shown in FIG. 14B, when the rotating body 61 rotates 45 degrees, the longitudinal direction of the cartridge 1 becomes parallel to a moving direction of the rod 82 of the pressing mechanism 80. The controller 90 drives the plunger motor 81 to move the rod 82. When the rod 82 comes into contact with the attachment stand 11A of the plunger 10 of the cartridge 1 and further moves, the plunger 10 is pushed into the tube 20 side. The controller 90 moves the rod 82 to the state shown in FIG. 14C and pushes the plunger 10 until the attachment stand 11A of the plunger 10 comes into contact with the upper edge of the tube 20.

When the plunger 10 is pushed into the tube 20 side, the seal 12A of the bar-like section 12 of the plunger 10 fits in the lower syringe 22 of the tube 20 (see FIG. 2B). When the plunger 10 is further pushed in, the seal 12A slides in the lower syringe 22. Consequently, the liquid (the third oil plug 48, the reaction solution plug 47, etc.) on the downstream side of the tube 20 is pushed out to the channel forming section 35 of the PCR container 30 by an amount equivalent to a volume of the slide of the seal 12A in the lower syringe 22.

First, the third oil plug 48 of the tube 20 flows into the channel forming section 35. Since the oil is filled in the channel forming section 35, the oil equivalent to the flown-in third oil plug 48 flows into the oil receiving space 32A from the channel forming section 35. The oil interface of the oil receiving space 32A rises. In this case, the pressure of the liquid in the channel forming section 35 is higher than the outside pressure (the pressure in the oil receiving space 32A) because of a pressure loss in the lower seal section 34B. After the third oil plug 48 is pushed out from the tube 20, the reaction solution plug 47 flows into the channel forming section 35 from the tube 20. Since the inner diameter of the channel forming section 35 is larger than the inner diameter of the capillary 23, the reaction solution plug 47 having a plug shape in the tube 20 changes to a droplet shape in the oil in the channel forming section 35.

The volume of the slide of the seal 12A in the lower syringe 22 (an amount of the liquid in the tube 20 pushed out from the downstream side) is larger than a total of the reaction solution plug 47 and the third oil plug 48 in the tube 20. Therefore, after the reaction solution plug 47 is pushed out from the tube 20, a part of the second oil plug 46 is also pushed out to the channel forming section 35. Consequently, the reaction solution does not remain in the tube 20. The entire liquid in the reaction solution plug 47 changes to a droplet shape. Since a part of the second oil plug 46 is pushed out from the downstream side of the tube 20, the reaction solution droplet 47 easily separates from the tube 20 (less easily adheres to the opening of the capillary 23).

The inner diameter of the terminal end of the capillary 23 (the opening diameter of the capillary 23) is designed relatively small, the reaction solution changed to the droplet in the PCR container 30 less easily adheres to the opening of the capillary 23. The specific gravity of the reaction solution is larger than the specific gravity of the oil in the PCR container 30. Therefore, the reaction solution droplet 47 separates from the terminal end of the capillary 23 and precipitates toward the bottom 35A using the channel forming section 35 as a channel. However, at this stage, since the channel of the channel forming section 35 tilts 45 degrees, the reaction solution droplet 47 easily adheres to the inner wall of the channel forming section 35. Therefore, it is necessary to return the channel of the channel forming section 35 to the vertical direction.

After the reaction solution droplet 47 is formed (after the plunger 10 is pushed), the controller 90 drives the plunger motor 81 in the opposite direction and returns the rod 82 to the original position. In this state, even if the cartridge 1 rotates, the rod 82 of the pressing mechanism 80 does not come into contact with the cartridge 1. In such a state, the controller 90 returns the rotating body 61 to the reference position. When the rotating body 61 is in the reference position, the channel of the channel forming section 35 changes to the vertical direction. Therefore, the reaction solution droplet 47 less easily adheres to the inner wall of the channel forming section 35.

### (4) Thermal Cycle Treatment

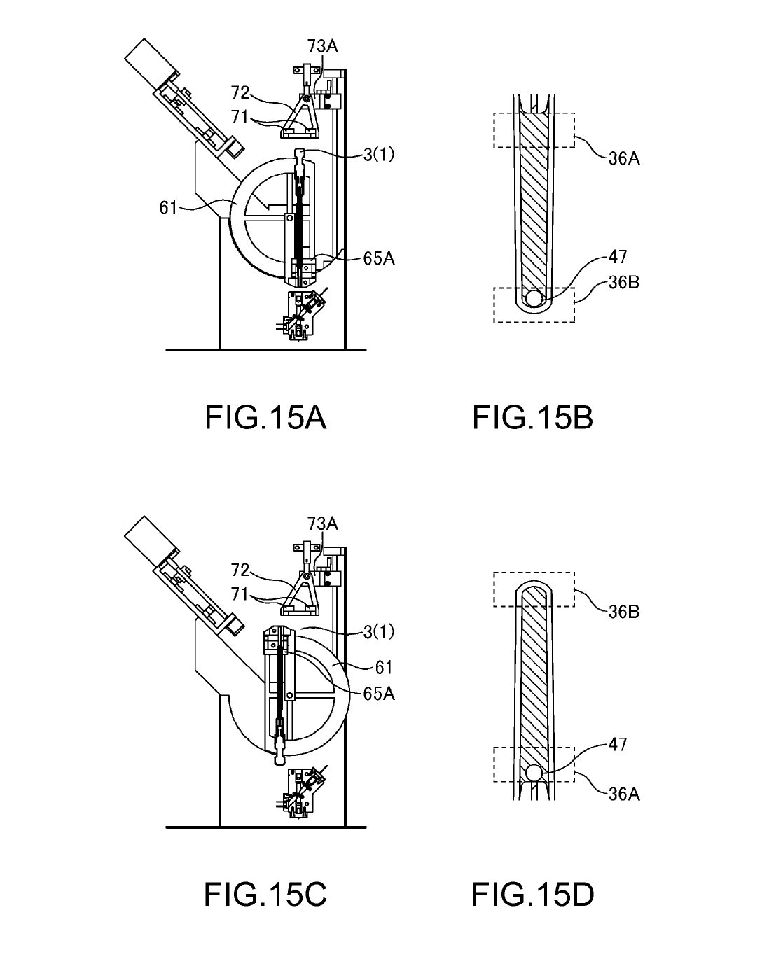

FIGS. 15A to 15D are explanatory diagrams of thermal cycle treatment. FIGS. 15A and 15B are explanatory diagrams of a state in which temperature treatment on a low-temperature side is applied to the reaction solution droplet 47. FIGS. 15C and 15D are explanatory diagrams of a state in which temperature treatment on a high-temperature side is applied to the reaction solution droplet 47. States of the PCR device 100 are shown in FIGS. 15A and 15C. States of the inside of the channel forming section 35 of the PCR container 30 are shown in FIGS. 15B and 15D.

When the cartridge 1 is fixed in the normal position with respect to the mounting section 62, the upstream side (the high-temperature region 36A) of the channel forming section 35 of the PCR container 30 is opposed to the high-temperature side heater 65B and the downstream side (the low-temperature region 36B) of the channel forming section 35 of the PCR container 30 is opposed to the low-temperature side heater 65C.

During the thermal cycle treatment, the controller 90 heats, with the high-temperature side heater 65B provided in the rotating body 61, liquid in the high-temperature region 36A on the upstream side of the channel forming section 35 of the PCR container 30 to about 90 to 100° C. The controller 90 heats, with the low-temperature side heater 65C provided in the rotating body 61, liquid in the low-temperature region 36B on the downstream side of the channel forming section 35 to about 50 to 75° C. Consequently, during the thermal cycle treatment, a temperature gradient is formed in the liquid in the channel forming section 35 of the PCR container 30. Since the mounting section 62 and the heaters are provided in the rotating body 61, even if the rotating body 61 rotates, the positional relation between the cartridge 1 and the heaters is maintained.

During the thermal cycle treatment, the liquid in the PCR container 30 is heated. If air bubbles occur when the liquid in the PCR container 30 is heated, it is likely that fluctuation occurs in the temperature of the liquid in the channel forming section 35 and movement (precipitation) of the reaction solution droplet 47 in the channel forming section 35 is inhibited. However, in this embodiment, since the pressure of the liquid in the channel forming section 35 is higher than the outside pressure because of a pressure loss in the lower seal section 34B, air bubbles less easily occur in the liquid in the PCR container 30.

When the rotating body 61 is present in the reference position as shown in FIG. 15A, as shown in FIG. 15B, the low-temperature region 36B (the low-temperature side heater 65C) is located on the lower side of the high-temperature region 36A (the high-temperature side heater 65B). The bottom 35A of the PCR container 30 of the cartridge 1 faces down. Since the specific gravity of the reaction solution droplet 47 is larger than the specific gravity of the oil, the reaction solution droplet 47 precipitates in the channel forming section 35. When the reaction solution droplet 47 precipitates in the channel forming section 35, the reaction solution droplet 47 reaches the bottom 35A of the PCR container 30. The reaction solution droplet 47 ends the precipitation on the bottom 35A and stays in the low-temperature region 36B. Consequently, the reaction solution droplet 47 moves to the low-temperature region 36B. The controller 90 keeps the state shown in FIG. 15B for a predetermined time and heats the reaction solution droplet 47 to about 50 to 75° C. in the low-temperature region 36B (the temperature treatment on the low-temperature side is applied). During this period, extension reaction of the polymerase reaction occurs.

When the controller 90 drives the rotating motor 66 to rotate the rotating body 61 180 degrees in the state shown in FIG. 15A, the state changes to the state shown in FIG. 15C. When the rotating body 61 rotates 180 degrees from the reference position, the cartridge 1 is vertically reversed. As shown in FIG. 15D, the high-temperature region 36A (the high-temperature side heater 65B) and the low-temperature region 36B (the low-temperature side heater 65C) are also vertically reversed. That is, the high-temperature region 36A (the high-temperature side heater 65B) is located on the lower side of the low-temperature region 36B (the low-temperature side heater 65C). The bottom 35A of the PCR container 30 of the cartridge 1 faces up. When the reaction solution droplet 47 precipitates in the channel forming section 35, the reaction solution droplet 47 reaches the terminal end of the tube 20 (the terminal end of the capillary 23). The reaction solution droplet 47 ends the precipitation at the terminal end of the tube 20 and stays in the high-temperature region 36A. Consequently, the reaction solution droplet 47 moves to the high-temperature region 36A. The controller 90 keeps the state shown in FIG. 15D for a predetermined time and heats the reaction solution droplet 47 to about 90 to 100° C. in the high-temperature region 36A (the temperature treatment on the high-temperature side is applied). During this period, degeneration reaction of polymerase reaction occurs.

When the controller 90 drives the rotating motor 66 to rotate the rotating body 61 −180 degrees in the state shown in FIG. 15C, the PCR container 30 of the cartridge 1 also rotates degrees and returns to the state shown in FIG. 15B. In this state, when the reaction solution droplet 47 precipitates in the channel forming section 35, the reaction solution droplet 47 moves to the low-temperature region 36B and is heated to about 50 to 75° C. again in the low-temperature region 36B (the temperature treatment on the low-temperature side is applied). Note that, since the inner diameter of the terminal end of the capillary 23 (the opening diameter of the capillary 23) is designed relatively small, the reaction solution droplet 47 less easily adheres to the opening of the capillary 23. Therefore, when the rotating body 61 rotates −180 degrees in the state shown in FIG. 15C (when the PCR container 30 rotates −180 degrees in the state shown in FIG. 15D), the reaction solution droplet 47 separates from the tube 20 and precipitates toward the bottom 35A of the PCR container 30 without adhering to the opening of the capillary 23.

The controller 90 repeats, at a predetermined cycle number, the driving of the rotating motor 66 and the changing of the rotating position of the rotating body 61 to the state shown in FIG. 15A and the state shown in FIG. 15C. Consequently, the PCR device 100 can apply the thermal cycle treatment of PCR to the reaction solution droplet 47.

In the storage device of the controller 90, thermal cycle information such as the temperature of the high-temperature side heater 65B, the temperature of the low-temperature side heater 65C, time for retention in the state shown in FIG. 15B, time for retention in the state shown in FIG. 15D, and a cycle number (the number of times of repetition of the state shown in FIG. 15B and the state shown in FIG. 15D) is stored. The controller 90 executes the processing explained above according to the thermal cycle information.

### (5) Fluorescence Measurement

As shown in FIG. 15A, when the rotating body 61 is present in the reference position, the fluorescence measuring instrument 55 is opposed to the bottom 35A of the PCR container of the cartridge 1. Therefore, during fluorescence measurement of the reaction solution droplet 47, the controller 90 causes the fluorescence measuring instrument 55 to measure fluorescence intensity of the reaction solution droplet 47 present on the bottom 35A of the PCR container 30 from the lower-side opening of the insertion hole 64A of the low-temperature side heater 65C in the state where the rotating body 61 is in the reference position.

Immediately after the rotating body 61 rotates 180 degrees to the reference position, the reaction solution droplet 47 is precipitating in the channel forming section 35 of the PCR container 30. The reaction solution droplet 47 sometimes does not reach the bottom 35A of the PCR container 30. Therefore, the controller 90 desirably measures the fluorescence intensity when a predetermined time elapses after the rotating position of the rotating body 61 changes to the state shown in FIG. 15A (immediately before the rotating body 61 is rotated in the state shown in FIG. 15A). Alternatively, the controller 90 may cause the fluorescence measuring instrument 55 to measure the fluorescence intensity for a predetermined time after the rotating body 61 is located in the reference position and store a time history of the fluorescence intensity.

### EXAMPLES

### Experimental Example 1

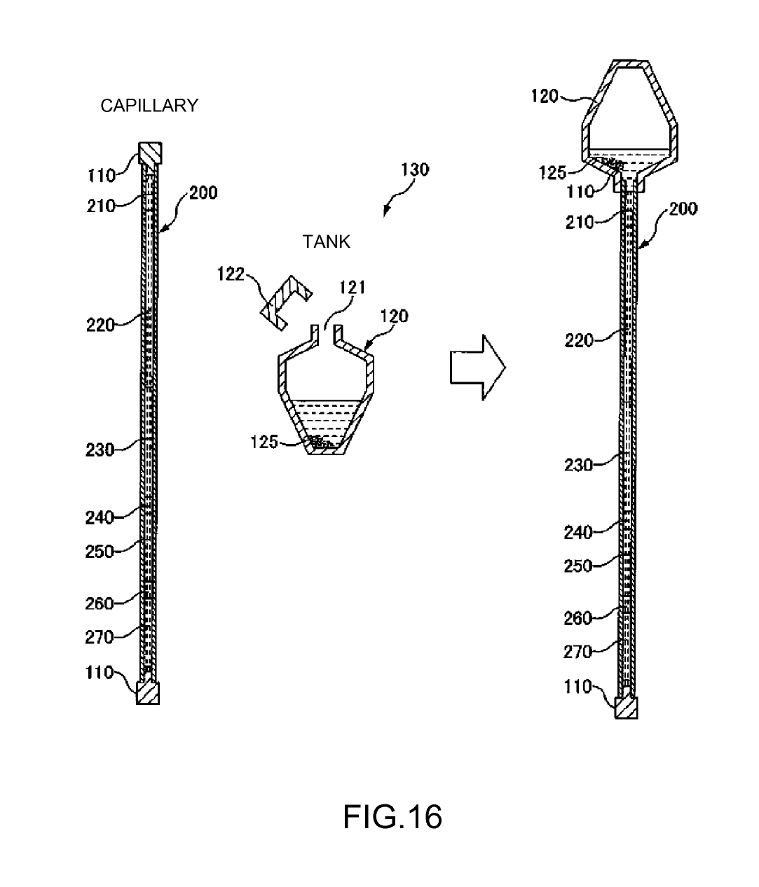

In this example, as shown in FIG. 16, a configuration in which a first plug 210 to a seventh plug 270 were included on the inside of a tube 200 in the kit for nucleic acid extraction was used.

First, 375 μL of an adsorbing solution and 1 μL of a magnetic bead dispersion solution were stored in a polyethylene container 130 having a capacity of 3 mL. As the adsorbing solution, a water solution of 76 mass % of guanidine hydrochloride, 1.7 mass % of disodium dihydrogen ethylenediaminetetraacetate dehydrate, and 10 mass % of polyoxyethylene sorbitan monolaurate (manufactured by Toyobo Co., Ltd., MagExtractor-Genome-, NPK-1) was used. As a magnetic bead stock solution, a stock solution containing 50 volume % of magnetic silica particles and 20 mass % of lithium chloride was used.

50 μL of blood collected from a human was put in a container 130 from a port 121 of the container 130 using a pipet. The container 130 was closed by a lid 122 and shaken by hand for 30 seconds to agitate the blood. Thereafter, the lid 122 of the container 130 was removed and the container 130 was connected to the tube 200. Stoppers 110 were formed at both ends of the tube 200. The stopper 110 on the first plug 210 side was removed and the container 130 was connected to the tube 200.

The first plug 210, the third plug 230, the seventh plug 270, and the fifth plug 250 were silicon oil. A cleaning solution A for the second plug 220 was a water solution of 76 mass % of guanidine hydrochloride. A cleaning solution B for the fourth plug 240 was a tris-hydrochloric acid buffer solution of pH 8.0 (solute concentration 5 mM). An eluate of the sixth plug 260 was sterilized water.

A permanent magnet 410 was moved to introduce magnetic beads 125 in the container 130 into the tube 200. The magnetic beads 125 were moved to the sixth plug 260. Times in which the magnetic beads 125 were present in the plugs in the tube 200 were approximately as described below. The first, third, and seventh plugs: 3 seconds each, the second plug: 20 seconds, the fourth plug: 20 seconds, and the sixth plug: 30 seconds. Note that, in the second plug 220 and the fourth plug 240, operation for, for example, vibrating the magnetic beads was not performed. The volumes of the second plug 220, the fourth plug 240, and the sixth plug 260 were respectively 25 μL, 25 μL, and 1 μL.

Subsequently, the stopper 110 on the seventh plug side of the tube was removed, the container 120 was deformed by hand and the seventh plug 270 and the sixth plug 260 were discharged to a reaction container of PCR. This operation was performed after the magnetic beads were moved by the permanent magnet and retracted to the second plug 220.

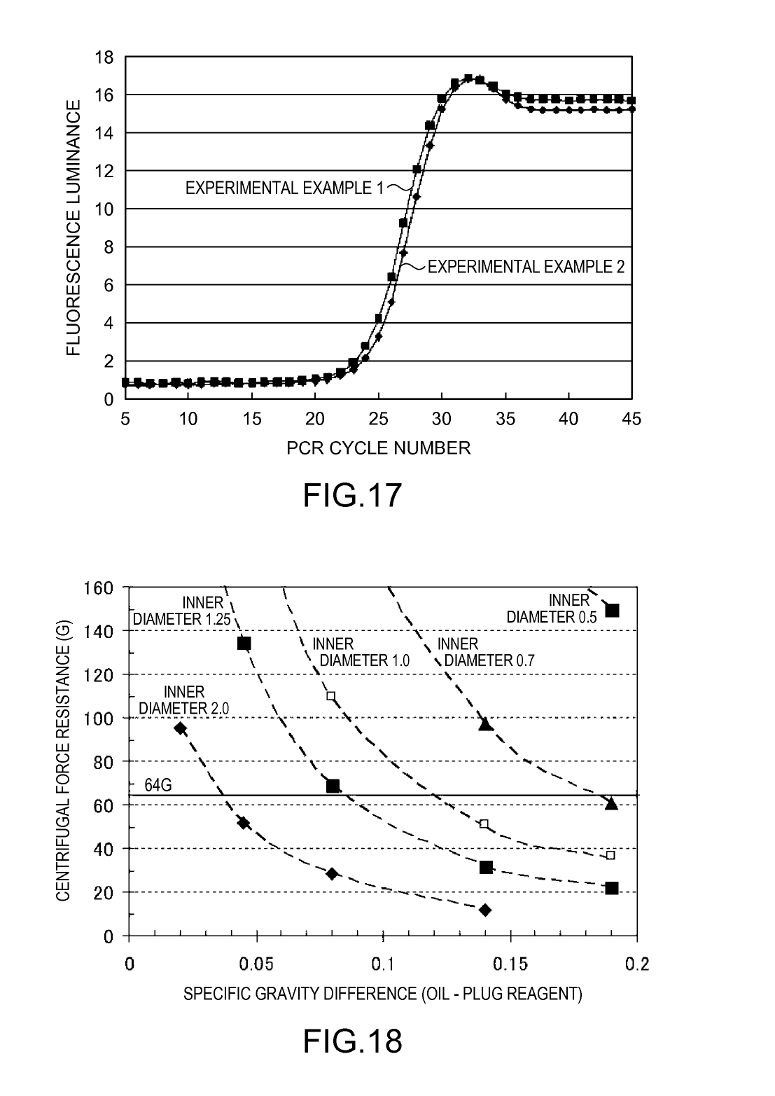

19 μL of a reaction reagent of PCR was added to an extracted solution of the plugs and real-time PCR was performed according to the rule. A breakdown of the reaction reagent of PCR is as follows: 4 μL of Light Cycler 480 Genotyping Master (manufactured by Roche Diagnostics K.K. 4 707 524), 0.4 μL of SYBR Green I (manufactured by Life Technologies Corporation S7563) diluted 1000 folds by sterilized water, 0.06 μL each of primers for 100 μM β actin detection (F/R), and 14.48 μL of sterilized water. An amplification curve of PCR in the experimental example 1 is shown in FIG. 17. Note that, in FIG. 17, the ordinate indicates fluorescence luminance and the abscissa indicates a cycle number of PCR.

### Experimental Example 2

In an experimental example 2, extraction of nucleic acid was performed by a general nucleic acid extraction method.

First, 375 μl of an adsorbing solution and 20 μL of a magnetic bead dispersion solution were stored in a polyethylene container (an Eppendorf tube) having a capacity of 1.5 mL. The composition of the adsorbing solution and the magnetic bead dispersion solution are the same as those in the experimental example 1.

50 μl of blood collected from a human was introduced into a container from a port of the container using a pipet. The container was closed by a lid and the blood was agitated for 10 minutes by a Vortex mixer. A magnetic stand and the pipet were operated to perform B/F separation operation. In this state, the magnetic beads and a small amount of the adsorbing solution remained in the container.

Subsequently, 450 μL of the cleaning solution A having a composition same as that in the experimental example 1 was introduced into the container. The container was closed by the lid and the solution in the container was agitated for 5 seconds by the Vortex mixer. The magnetic stand and the pipet were operated to remove the cleaning solution A. This operation was repeated twice. In this state, the magnetic beads and a small amount of the cleaning solution A remained in the container.

Subsequently, 450 μL of the cleaning solution B having a composition same as that in the experimental example 1 was introduced into the container. The container was closed by the lid and the solution in the container was agitated for 5 seconds by the Vortex mixer. The magnetic stand and the pipet were operated to remove the cleaning solution B. This operation was repeated twice. In this state, the magnetic beads and a small amount of the cleaning solution B remained in the container.

50 μL of sterilized water (an eluate) was added to the container. The container was closed by the lid and the solution in the container was agitated for 10 minutes by the Vortex mixer. The magnetic stand and the pipet were operated to collect supernatant liquid. The supernatant liquid contains target nucleic acid.

1 μL was dispensed from the extracted solution, 19 μL of a reaction reagent of PCR was further added, and the real-time PCR was performed according to the rule. A breakdown of the reaction reagent of PCR is as follows: 4 μL of Light Cycler 480 Genotyping Master (manufactured by Roche Diagnostics K.K. 4 707 524), 0.4 μL of SYBR Green I (manufactured by Life Technologies Corporation S7563) diluted 1000 folds by sterilized water, 0.06 μL each of primers for 100 μM β actin detection (F/R), and 14.48 μL of sterilized water. An amplification curve in this case is shown in FIG. 17.

### Experimental Results

Understanding explained below can be obtained from the experimental examples.

(1) When time required for the extraction treatment for nucleic acid, which is pre-treatment of PCR, is compared, time from the insertion of a specimen into the container until the target nucleic acid is introduced into the reaction container of PCR was about 2 minutes in the experimental example 1 and was about 30 minutes in the experimental example 2. Therefore, the time required for nucleic acid extraction is greatly shorter in the nucleic acid extraction method of the experimental example 1 than the nucleic acid extracting method of the experimental example 2.

(2) Amounts of the cleaning solutions in the experimental example 1 were about 1/18 of amounts of the cleaning solutions in the experimental example 2. Further, an amount of the eluate in the experimental example 1 was about 1/50 of an amount of the eluate in the experimental example 2. Therefore, in the experimental example 1, extremely small amounts of the cleaning solutions and the eluate were sufficient compared with the experimental example 2.

(3) When the concentration of the target nucleic acid in the eluate is compared in amounts of the adsorbing solution and the eluate, ideally, the concentration in the experimental example 1 is considered to be fifty times as large as the concentration in the experimental example 2. However, in the experimental examples, a nucleic acid amount contained in a blood sample is large and exceeds an adsorbable amount of 1 μL, of magnetic beads. An entire amount of nucleic acid contained in the blood sample cannot be collected. Therefore, in the experimental example 1, the concentration is not fifty times as large as the concentration in the experimental example 2. In the case of a specimen in which a nucleic acid content is small and does not exceed the adsorbable amount of 1 μL of magnetic beads, in the experimental example 1, the concentration is fifty times as large as the concentration in the experimental example 2.

(4) Referring to a graph of FIG. 17, in all blood samples with large contents of nucleic acid, a rising edge of an amplification factor of nucleic acid is about 0.6 cycle earlier in the experimental example 1 than in the experimental example 2. That is, the reaction solution of PCR used in the experimental example 1 has the concentration of the target nucleic acid higher than the concentration of the target nucleic acid in the reaction solution of PCR used in the experimental example 2. That is, the concentration of the target nucleic acid in the eluate is higher in the experimental example 1 than in the experimental example 2.

### Experimental Example 3

In this experimental example, it was examined how a specific gravity difference between a solution plug and an oil plug affected shock resistance. The shock resistance was specified as drop impact acceleration G and centrifugal force resistance.

First, to check which degree of shock resistance is necessary, the drop impact acceleration G was calculated. Specifically, when the drop impact acceleration G (=v/gt) applied to a cartridge having weight of 3.0 g when the cartridge was dropped from height of 2 meters (a collision time was assumed to be 0.1 second) was calculated using the following expression, the drop impact acceleration G was 64 G.

G[unit is G]=F/gm [Expression]

where, a drop impact force F can be calculated by the following expression.

F[unit is N]=(m/t)×√(2gh) [Expression]

(m: weight [kg], t: impact time [second], g=9.8 [m/s²], h: height [m])

Subsequently, a degree of shock resistance of an actual capillary was measured as centrifugal force resistance. Capillaries made of polypropylene having inner diameters of 0.5 mm, 0.7 mm, 1.0 mm, 1.25 mm, and 2.0 mm and length of about 30 mm were prepared. A plug (having length of 5 mm) made of pure water was created and oil plugs were created on both sides of the plug. Both ends of the plug were closed. As oils, silicone oils were used. The silicone oils were prepared such that specific gravity differences from pure water were 0.02, 0.045, 0.08, 0.14, and 0.19. The plugs were put in a tube and centrifuged by a centrifugal machine for 30 seconds such that a predetermined centrifugal force was applied to the plugs. A minimum centrifugal force with which the pure water plug moved was specified as centrifugal force resistance. Obtained results are shown in Table 1 and shown in FIG. 18 as a graph.

TABLE 1

| Specific | Inner | Specific | Inner | Specific | Inner | Specific | Inner | Specific | Inner |
|---|---|---|---|---|---|---|---|---|---|
| gravity | diameter | gravity | diameter | gravity | diameter | gravity | diameter | gravity | diameter |
| difference | 0.5 | difference | 0.7 | difference | 1.0 | difference | 1.25 | difference | 2.0 |
| 0.02 | NT | 0.02 | NT | 0.02 | NT | 0.02 | 243.2 | 0.02 | 95 |
| 0.045 | NT | 0.045 | 420 | 0.045 | 210 | 0.045 | 134.4 | 0.045 | 52 |
| 0.08 | 450 | 0.08 | 215 | 0.08 | 108 | 0.08 | 69.12 | 0.08 | 28 |
| 0.14 | 210 | 0.14 | 97 | 0.14 | 50 | 0.14 | 32 | 0.14 | 12 |
| 0.19 | 150 | 0.19 | 61 | 0.19 | 35 | 0.19 | 22.4 | 0.19 | NT |

(NT: note measured)

In this way, irrespective of the inner diameters of the tubes in use, the shock resistance was larger when the specific gravity difference was smaller.

To set the centrifugal force resistance to be equal to or smaller than 64 G, which is drop impact acceleration predicted during use, when the inner diameter was 1.0 mm, the specific gravity difference needed to be equal to or smaller than about 0.12.

### Experimental Example 4

In this experimental example, it was examined how a specific gravity difference between a solution plug and an oil plug in a PCR container affected a moving time of a droplet.

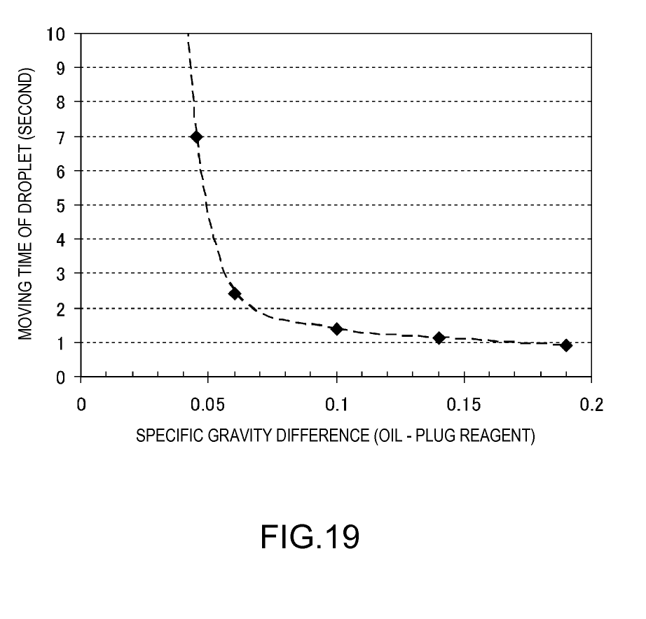

Silicon oil was filled in a PCR container having an inner diameter of 2.0 mm and length of 25.0 mm. Droplets formed by 1 μl of pure water were created in the PCR container. Droplets and silicon oils having specific gravity differences of 0.045, 0.06, 0.1, 0.14, and 0.19 were used to measure times in which the droplets dropped in the oils. Obtained results are shown in FIG. 19. Note that the silicon oils were prepared in the same manner as in the experimental example 1.

As it is seen from FIG. 19, the moving speed of the droplet in the oil was larger as the specific gravity difference was larger.

When PCR was actually performed by the elevating PCR device explained above, a moving time of the droplet for completing reaction of 40 cycles within 10 minutes was equal to or shorter than 2.5 seconds. A specific gravity difference with which this time was obtained was equal to or larger than 0.06.

The entire disclosure of Japanese Patent Application No. 2013-185679, filed Sep. 6, 2013 is expressly incorporated by reference herein.

What is claimed is:

1. A cartridge for nucleic acid amplification reaction comprising:
a tube including, on an inside, in order, a first plug formed by first oil, a second plug formed by a first cleaning solution, which causes phase separation when mixed with the first oil or second oil and cleans a nucleic acid binding solid-phase carrier bound with nucleic acid, a third plug formed by the second oil, a fourth plug formed by an eluate, which causes phase separation when mixed with the second oil or third oil and elutes the nucleic acid from the nucleic acid binding solid-phase carrier bound with the nucleic acid, and a fifth plug formed by the third oil; and
a container for nucleic acid amplification reaction communicating with the fifth plug side of the tube and including fourth oil on an inside, wherein
at least one of the first to third oils and the fourth oil have different specific gravities.

2. The cartridge for nucleic acid amplification reaction according to claim 1, further comprising a plunger attached to an opening section on the first plug side of the tube and configured to push out liquid from the fifth plug side of the tube to the container for nucleic acid amplification reaction, wherein
on an inside of the plunger, fifth oil and a second cleaning solution, which causes phase separation when mixed with the first oil or the fifth oil and cleans the nucleic acid binding solid-phase carrier bound with the nucleic acid are stored.

3. The cartridge for nucleic acid amplification reaction according to claim 1, wherein the eluate contains a reagent that causes reverse transcription reaction.

4. The cartridge for nucleic acid amplification reaction according to claim 3, wherein the eluate contains a reagent that causes nucleic acid amplification reaction.

5. The cartridge for nucleic acid amplification reaction according to claim 1, wherein the container for nucleic acid amplification reaction includes a seal forming section configured to fix the tube and a channel forming section in which droplets move.

6. The cartridge for nucleic acid amplification reaction according to claim 5, wherein the seal forming section includes an oil receiving section configured to receive oil overflowing the channel forming section.

7. The cartridge for nucleic acid amplification reaction according to claim 1, further comprising a tank communicating with the first plug side of the tube and configured to introduce the nucleic acid binding solid-phase carrier into the tube.

8. The cartridge for nucleic acid amplification reaction according to claim 7, wherein the tank and the tube are combined via the plunger.

9. The cartridge for nucleic acid amplification reaction according to claim 1, wherein the fourth oil has the specific gravity smaller than specific gravities of all of the first to third oils.

10. The cartridge for nucleic acid amplification reaction according to claim 1, wherein, when specific gravities of the first to third oils are respectively represented as A₁ to A₃ and specific gravities of the first cleaning solution and the eluate are respectively represented as B₁ and B₂, concerning the plugs adjacent to each other and concerning at least one of A₁ to A₃ and at least one of B₁ and B₂, |Bm−An|≦0.12 (in the formula, n is an integer of 1 to 3 and m is 1 or 2) holds.

11. The cartridge for nucleic acid amplification reaction according to claim 1, wherein, when specific gravity of the fourth oil is represented as A₄ and specific gravities of the eluate is represented as B₂, |B₂−A₄|≧0.06 holds.

12. The cartridge for nucleic acid amplification reaction according to claim 1, wherein specific gravities of the first to third oils are equal to or larger than 0.88 and equal to or smaller than 1.10, specific gravity of the fourth oil is equal to or larger than 0.80 and equal to or smaller than 0.95, and specific gravities of the cleaning solution and the eluate are equal to or larger than 1.00 and equal to or smaller than 1.20.

13. A cartridge kit for nucleic acid amplification reaction comprising:
the cartridge for nucleic acid amplification reaction according to claim 1; and
a tank configured to introduce the nucleic acid binding solid-phase carrier into the tube.

14. A cartridge for nucleic acid amplification reaction comprising:
a tube including, on an inside, in order, a first plug formed by an eluate that elutes nucleic acid from a nucleic acid combining solid-phase carrier bound with the nucleic acid and a second plug formed by first oil, which causes phase separation when mixed with the eluate; and
a container for nucleic acid amplification reaction communicating with the second plug side of the tube and including second oil on an inside, wherein
the first oil and the second oil have different specific gravities.

15. The cartridge for nucleic acid amplification reaction according to claim 14, wherein the tube includes, on an inside, a third plug formed by third oil, which causes phase separation when mixed with the eluate on a side of the first plug.

16. The cartridge for nucleic acid amplification reaction according to claim 15, wherein the second oil has specific gravity smaller than both the specific gravities of the first and third oils.
